SEQUENCE LISTING (13 sequences)

SEQ ID NO: 1 - protein, 202 aa, Homo sapiens
MDCQLSILLLLSCSVLDSFGELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKC

SEQ ID NO: 2 - protein, 5 aa, Artificial Sequence
  VH region CDR1
GYWMN

SEQ ID NO: 3 - protein, 17 aa, Artificial Sequence
  VH region CDR2
DIYPGSGNTNYDEKFQG

SEQ ID NO: 4 - protein, 9 aa, Artificial Sequence
  VH region CDR3
GGYYEDFDS

SEQ ID NO: 5 - protein, 10 aa, Artificial Sequence
  VL region CDR1
RASQGIISYL

SEQ ID NO: 6 - protein, 7 aa, Artificial Sequence
  VL region CDR2
AASSLQS

SEQ ID NO: 7 - protein, 9 aa, Artificial Sequence
  VL region CDR3
GQYANYPYT

SEQ ID NO: 8 - protein, 19 aa, Artificial Sequence
  KB004 Heavy chain signal sequence
MEWSWVFLFFLSVTTGVHS

SEQ ID NO: 9 - protein, 20 aa, Artificial Sequence
  KB004 Light chain signal sequence
MSVPTQVLGLLLLWLTDARC

SEQ ID NO: 10 - protein, 118 aa, Artificial Sequence
  KB004 VH region sequence
QVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSS

SEQ ID NO: 11 - protein, 107 aa, Artificial Sequence
  KB004 VL region sequence
DIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIK

SEQ ID NO: 12 - protein, 448 aa, Artificial Sequence
  KB004 Heavy chain sequence
QVQLVQSGAEVKKPGASVKVSCKASGYTFTGYWMNWVRQAPGQGLEWMGDIYPGSGNTNYDEKFQGRVTMTRDTSISTAYMELSRLRSDDTAVYYCARGGYYEDFDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial Sequence
  KB004 Kappa chain sequence
DIQMTQSPSFLSASVGDRVTITCRASQGIISYLAWYQQKPEKAPKRLIYAASSLQSGVPSRFSGSGSGTEFTLTISSLQPEDFATYYCGQYANYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0270821 A1
(43) Pub. Date: Sep. 5, 2019

(54) EPHA3 ANTIBODIES FOR THE TREATMENT OF PULMONARY FIBROSIS

(71) Applicant: HUMANIGEN, INC, Burlingame, CA (US)

(72) Inventor: Geoffrey T YARRANTON, Burlingame, CA (US)

(73) Assignee: HUMANIGEN, INC., Burlingame, CA (US)

(21) Appl. No.: 16/333,094

(22) PCT Filed: Sep. 13, 2017
(86) PCT No.: PCT/US17/51406

§ 371 Date: Mar. 13, 2019

Related U.S. Application Data
(60) Provisional application No. 62/394,097, filed on Sep. 13, 2016.

(51) Int. Cl. C07K 16/28; A61P 11/00; A61K 9/00; G01N 33/533; G01N 33/68

(52) U.S. Cl. CPC C07K 16/2863; A61P 11/00; G01N 33/6884; G01N 33/533; A61K 9/0019

(57) ABSTRACT

The invention provides methods of treating or preventing pulmonary fibrosis or idiopathic pulmonary fibrosis (IPF) by administering an anti-EphA3 antibody to a patient. Moreover, the invention provides methods of identifying a candidate for treatment with an anti-EphA3 antibody by evaluating BAL fluid for EphA3 positivity are also disclosed. The invention further provides methods of measuring the efficacy of treatment by evaluating the reduction of the disease markers of fibrosis in a pulmonary fibrosis or IPF patient treated with an anti-EphA3 antibody are also disclosed.

### FIELD OF TECHNOLOGY

The invention provides methods of treating or preventing pulmonary fibrosis or idiopathic pulmonary fibrosis (IPF) by administering an anti-EphA3 antibody to a patient. Moreover, the invention provides methods of identifying a candidate for treatment with an anti-EphA3 antibody by evaluating bronchoalveolar lavage (BAL) fluid for EphA3 positivity are also disclosed. The invention further provides methods of measuring the efficacy of treatment by evaluating the reduction of the disease markers of fibrosis in a pulmonary fibrosis or IPF patient treated with an anti-EphA3 antibody are also disclosed.

### BACKGROUND

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) control cell positioning and tissue organization (Poliakov. A., et al., Dev Cell 7:465-80 (2004)). In contrast to other receptor tyrosine kinases, Eph receptor activation does not only require ligand binding and dimerization, but also involves preformed ligand oligomers. Thus, tyrosine phosphorylation of Eph receptors requires presentation of ephrin ligands in their clustered or membrane-attached forms (Davis et al., Science 266:816-819 (1994)). Functional and biochemical Eph responses occur at higher ligand oligomerization states (Stein et al., Genes Dev 12:667-678 (1998)).

Among other patterning functions, various Ephs and ephrins have been shown to play a role in vascular development. The de-regulated re-emergence of some ephrins and their receptors in adults also has been observed to contribute to tumor invasion, metastasis and neo-angiogenesis. EphA3 has been shown to be overexpressed in tumor stromal cells and vascular endothelium in solid tumors. Further, EphA3 is expressed in aberrant vasculature, bone marrow stem cells, and stromal tissues in various hematological disorders, including myelofibrosis, polycythemia vera, myelodysplastic syndrome and essential thrombocythemia; and in chronic and acute myeloid leukemias. It is also upregulated in diseases characterized by abnormal proliferation and fibrosis, such as idiopathic pulmonary fibrosis and diabetic kidney disease.

Idiopathic pulmonary fibrosis (IPF) is a form of chronic, progressive fibrosing interstitial pneumonia of unknown cause and associated with the histopathologic and/or radiologic pattern of usual interstitial pneumonia (UIP). It has been proposed that IPF results from an aberrant activation of alveolar epithelia cells and mesenchymal cells leading to accumulation of extracellular matrix and irreversible destruction of the lung parenchyma.

KB004 is a Humaneered® high affinity antibody (K_{D}=about 610 pM) targeting EphA3 with at least 3 possible mechanisms of action: direct apoptosis in tumor cells, activation of ADCC and disruption of tumor vasculature. It has been proposed as a therapeutic agent for the treatment of hematological malignancies and solid tumors.

### SUMMARY

The present invention provides a method for treating or preventing pulmonary fibrosis, e.g., IPF, the method comprising administering an anti-EphA3 antibody to a patient that has pulmonary fibrosis is or at risk for having pulmonary fibrosis. In some embodiments, the anti-EphA3 antibody induces ADCC and/or activates EphA3, e.g., KB004 or an antibody having the binding specificity of KB004. In some embodiments, KB004, or an antibody having the binding specificity of KB004 is administered to a patient that has IPF.

In one aspect, disclosed is a method treating or preventing pulmonary fibrosis, the method comprising administering an effective amount of an anti-EphA3 antibody to a patient having pulmonary fibrosis or to a patient at risk for pulmonary fibrosis. In one aspect, disclosed is a method treating or preventing IPF, the method comprising administering an effective amount of an anti-EphA3 antibody to a patient having IPF or to a patient at risk for IPF.

In another aspect, disclosed is a use of an anti-EphA3 antibody for the manufacture of a medicament for the treatment or prevention of pulmonary fibrosis, wherein the medicament is administered in an effective amount to a patient having pulmonary fibrosis or to a patient at risk for IPF. In one aspect, disclosed is a use of an anti-EphA3 antibody for the manufacture of a medicament for the treatment or prevention of IPF, wherein the medicament is administered in an effective amount to a patient having IPF or to a patient at risk for IPF.

In one aspect, disclosed are methods of identifying a candidate for treatment with an anti-EphA3 antibody by evaluating bronchoalveolar lavage (BAL) fluid for EphA3 positivity. In another aspect, disclosed are methods of measuring the efficacy of treatment by evaluating the reduction of the disease markers of fibrosis in a pulmonary fibrosis or IPF patient treated with an anti-EphA3 antibody.

### BRIEF DESCRIPTION OF THE DRAWINGS

The compositions, methods and uses disclosed herein may best be understood by reference to the following detailed description when read with the accompanying drawings in which:

FIG. 1 shows that EphA3 transcripts are expressed in primary human fibroblast cultures from IPF.

FIG. 2 shows EphA3 Staining of Fibrotic Lung in IPF.

FIG. 3 shows the experiments design to evaluate the efficacy of KB004 in treating IPF.

FIG. 4 provides data illustrating that KB004 treatment resulted in reduced lung fibrosis compared to control immunoglobulin G (IgG) (KB00243). Histological analysis was performed by trichrome staining at day 35.

FIG. 5 provides data illustrating that hydroxyproline (fibrosis) levels in the lungs of KB004 vs. control IgG-treated mice were reduced.

FIG. 6A-FIG. 6F provide data illustrating that disease-associate markers are reduced in cells in lungs of KB004-treated vs. control-treated mice.

FIG. 7A-FIG. 7B show the high levels of EphA3 in idiopathic pulmonary fibrosis (IPF) lung biopsies (FIG. 7A) and IPF lung explants (FIG. 7B).

FIG. 8A-FIG. 8B show the levels of EphA3 in lung disease other than IPF such as COPD lungs (FIG. 8A) and pulmonary adenocarcinoma (FIG. 8B).

FIG. 9A-FIG. 9I provide hematologic data illustrating that stage-specific embryonic antigen 4 (SSEA-4⁺) stromal cell progenitors in an IPF lung are EphA3 positive compared to normal lungs. FIG. 9A shows the florescent staining of SSEA-4⁺ stromal cell progenitors in a normal lung. FIG. 9B shows the florescent staining of SSEA-4⁺ stromal cell progenitors in an idiopathic pulmonary fibrosis (IPF) lung. Scatterplots of the detection of CD45 and EpCAM markers in SSEA-4⁺ cells are shown for normal lungs (FIG. 9C), slow progression IPF (FIG. 9D), and rapid progression IPF (FIG. 9E). The data illustrated in FIG. 9F-FIG. 9I show the concentration of CD45+ and EpCAM expressing EphA3.

FIG. 10A-FIG. 10E show the regulation of EphA3 in fibroblast/progenitor cultures. FIG. 10A provides data showing the percent fold change, above untreated, of cells expressing EphA3 on the cell surface. FIG. 10B provides flow cytometry data showing the concentrations of EphA3 normalized to mode for untreated (Unt), GpC, and interleukin 1 beta (IL1β) cells. FIG. 10C-FIG. 10E show the side scatter light flow cytometry plots for untreated (FIG. 10C). GpC (FIG. 10D), and IL1 L1β (FIG. 10E).

FIG. 11 shows the experimental design for measuring the role of EphA3 in freshly isolated IPF lung explant cellular suspensions.

FIG. 12A-FIG. 12H show the flow cytometric characterization of IPF lung explant cellular suspensions for a variety of markers.

FIG. 13 shows lung explant cells 9 days post culture which have fibroblast precursors.

FIG. 14A-FIG. 14C show the appearance of fibroblast colonies 9 days post culture in cells that were untreated (FIG. 14A), treated with 20 μg/ml of KB004 once every three days (FIG. 14B), and treated with 20 μg/ml of KB1623D once every three days (FIG. 14C).

FIG. 15A-FIG. 15B provide data illustrating the transcript analysis after 9 days of culture and treatment with a profibrotic panel (FIG. 15A) and neuroendocrine panel (FIG. 15B).

FIG. 16 shows the experimental design for modeling IPF in NOD/SCID mice.

FIG. 17A-FIG. 17D show histological analysis of fibrosis in SCID mice 63 days after injection. FIG. 17A-FIG. 17B show lung tissue samples taken from naïve lungs (FIG. 17A) and IPF lungs (FIG. 17B). FIG. 17C-FIG. 17D show spleen tissue samples taken from naïve spleen (FIG. 17C) and IPF spleens (FIG. 17D).

FIG. 18 provides data illustrating that hydroxyproline (fibrosis) levels in the naïve versus IPF mice.

FIG. 19A-FIG. 19B show the spleen of mice 63 days after injection with saline (FIG. 19A) and IPF cells (FIG. 19B).

FIG. 20A shows the histological analysis of naïve explant cells. FIG. 20B shows the flow cytometry of CD90 versus CD73 markers.

FIG. 21A shows the histological analysis of stromal cells. FIG. 21B shows the flow cytometry of CD90 versus CD73 markers.

FIG. 22 shows the experimental design for detecting EphA3+stromal cells in murine stromal cultures.

FIG. 23A-FIG. 23D show flow cytometric analysis of stromal cultures developed from cellular suspensions of murine lungs post explant cell challenge. FIG. 23A-FIG. 23B provide flow cytometry data showing the concentrations of CD90 (FIG. 23A) and CD73 (FIG. 23B) markers in the isotype cultures. FIG. 23C-FIG. 23D provide flow cytometry data showing the concentration of CD90 markers and CD73 markers in cultures from mouse 1 (FIG. 23C) and mouse 2 (FIG. 23D).

FIG. 24 shows the experimental design for measuring the efficacy of KB004 as a preventative intervention in a humanized NOD/SCID model of IPF.

FIG. 25A-FIG. 25C show the histological analysis at day 35 of mice of the preventative intervention experiment approach that were administered saline (FIG. 25A), KB00243 (FIG. 25B), and KB004 (FIG. 25C). FIG. 25-FIG. 25E show the histological analysis at day 63 of mice of the preventative intervention experiment approach that were administered KB00243 (FIG. 25D) and KB004 (FIG. 25E).

FIG. 26 provides data comparing the levels of hydroxyproline (Hyp) on day 35 and day 63 in mice that were administered saline, KB00243, and KB004.

FIG. 27A-FIG. 27B provides data illustrating the levels of CD45+, CCR10, and EPCAM markers in the lung (FIG. 27A) and spleen (FIG. 27B) on day 35 of mice that were administered KB00243 and KB004.

FIG. 28A-FIG. 28B provides data illustrating the levels of CD45+, CCR10, and EPCAM markers in the lung (FIG. 27A) and spleen (FIG. 27B) on day 63 of mice that were administered KB00243 and KB004.

FIG. 29 shows the experimental design for measuring the efficacy of KB004 as a therapeutic intervention in a humanized NOD/SCID model of IPF.

FIG. 30A-FIG. 30B show the histological analysis at day 35 of mice of the preventative intervention experiment approach that were administered KB00243 (FIG. 30A) and KB004 (FIG. 30B).

FIG. 31 provides data comparing the levels of hydroxyproline (Hyp) on day 35 in mice that were administered KB00243 and KB004.

FIG. 32A-FIG. 32B provides flow cytometry analysis showing the concentrations of EPCAM and CCR10 in the spleen of mice treated with KB00243 (FIG. 32A) and KB004 (FIG. 32B). FIG. 32C shows the percent of cells positive for CD45+ on day 35 in the spleen.

FIG. 33A-FIG. 33B provide flow cytometry analysis showing the concentrations of EPCAM and CCR10 in the lung of mice treated with KB00243 (FIG. 33A) and KB004 (FIG. 33B). FIG. 33C shows the percent of cells positive for CD45+ on day 35 in the spleen.

FIG. 34A-FIG. 34B provide flow cytometry data showing the concentrations of CD3 and SSC-A markers in the spleen (FIG. 34A) and lung (FIG. 34B) 63 days post explant cell challenge.

FIG. 35A-FIG. 35A show the bronchoalveolar lavage fluid (BALF) levels 63 days post explant cell challenge. FIG. 35C shows the serum levels 63 days post explant cell challenge.

FIG. 36A-FIG. 36D provide flow cytometry analysis showing the concentrations of EPCAM and CCR10.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

Pulmonary fibrosis is a condition in which tissue in the lungs becomes scarred over time. Causes of pulmonary fibrosis include environmental pollutants, some connective tissue diseases, and interstitial lung disease.

In one embodiment, the term “idiopathic pulmonary fibrosis” refers to a non-neoplastic pulmonary disease that is characterized by the formation of scar tissue within the lungs in the absence of any known provocation. IPF is a rare disease which affects approximately 5 million persons worldwide. The age at presentation is usually between 50-70 years. Clinical presentation includes progressive dyspnea on exertion, paroxysmal cough, usually nonproductive, abnormal breath sounds, abnormal chest x-ray or HRCT, and restrictive pulmonary physiology. For example, IPF initially manifests with symptoms of exercise-induced breathless and dry coughing. Auscultation of the lungs reveals early inspiratory crackles, predominantly located in the lower posterior lung zones upon physical exam. Digital clubbing is found in approximately 25%-50% of IPF patients. PF is recognized on high-resolution computed tomography by peripheral, subpleural lower lobe reticular opacities in association with subpleural honeycomb changes. IPF is also associated with a pathological lesion known as usual interstitial pneumonia (UIP). The UIP pattern consists of normal lung alternating with patches of dense fibrosis, taking the form of collagen sheets. Pulmonary hypertension at rest may also be observed (20%-40% of patients). Surgical lung biopsy is also used for diagnosing IPF.

The term “EphA3+” when used in the context of expression on fibrotic cells in IPF refers to EphA3 expression that is above background.

In one embodiment, the term “EphA3” refers to the Eph receptor A3. This receptor has also been referred to as “Human embryo kinase”. “hek”, “eph-like tyrosine kinase 1”, “etk1” or “tyro4”. EphA3 belongs to the ephrin receptor subfamily of the protein-tyrosine kinase family. EPH and EPH-related receptors have been implicated in mediating developmental events. Receptors in the EPH subfamily typically have a single kinase domain and an extracellular region containing a Cys-rich domain and 2 fibronectin type III repeats. The ephrin receptors are divided into 2 groups based on the similarity of their extracellular domain sequences and their affinities for binding ephrin-A and ephrin-B ligands. EphA3 binds ephrin-A ligands. EphA3 nucleic acid and protein sequences are known. An example of a human EphA3 amino acid sequence is available under accession number EAW68857.

In the context of this invention, “an anti-EphA3 antibody that activates EphA3 or induces ADCC” refers to an antibody that (i) activates EphA3 (ii) induces ADCC, or (iii) activates and induces ADCC.

For the purposes of the present invention, “activation” of EphA3 causes phosphorylation of EphA3. An antibody that activates EphA3 or “an activating antibody” causes phosphorylation of EphA3 and shape change, e.g., rounding, of endothelial cells and is therefore considered to be an agonist in the context of this invention. EphA3 can be activated by dimerization, which leads to shape change, e.g., rounding, in endothelial cells and may lead to apoptosis. In some embodiments, an antibody that activates EphA3 competes with mAb IIIA4 for binding to EphA3 (see. U.S. Pat. No. 8,664,365, incorporated by reference, for the CDRs of mAbIIIA4 and illustrative antibodies that compete for binding with mAbIIIA4). Typically, an “activating” antibody binds to the ligand binding domain (amino acids 29-202 of EphA3) wherein amino acid residues 131, 132, and 136 are important for binding. In some embodiments, the activating antibody binds to a site encompassing the residues 131, 132, and 136 within the ligand binding domain of human EphA3 protein.

In one embodiment, the terms “EphA3 antibody” or “anti-EphA3 antibody” are used interchangeably to refer to an antibody that specifically binds to EphA3. In some embodiments, the antibody can dimerize EphA3. The term encompasses antibodies that bind to EphA3 in the presence of ephrin ligand (e.g., ephrin-A5) binding, as well as antibodies that bind to the ligand binding site.

In one embodiment, the phrase “EphA3 antibody that binds to EphA3 in the presence of binding of an ephrin ligand” refers to an antibody that does not significantly prevent binding of an ephrin ligand, such as ephrin-A5, to EphA3. The presence of such an antibody in a binding reaction comprising EphA3 and an ephrin ligand, e.g., ephrin-A5, reduces ephrin ligand binding to EphA3 by less than about 30%, typically less than 20% or 10%.

In one embodiment, the term “KB004” refers to a non-fucosylated monoclonal antibody characterized by the heavy chain and light chain sequences show in the Table of KB004 sequences. “KB004” has ADCC activity and activates EphA3 upon binding. An antibody having the binding specificity of KB004 binds to EphA3 in the presence of an ephrin binding ligand and competes with KB004 for binding to EphA3. Illustrative examples of such antibodies are provided in U.S. Pat. No. 8,664,365, which is incorporated by reference.

In one embodiment, the phrase “antibody having an active isotype” refers to an antibody that has a human Fc region that binds to an Fc receptor present on immune effector cells. “Active isotypes” include IgG1, IgG3, IgM, IgA, and IgE. The term encompasses antibodies that have a human Fc region that comprises modifications, such as mutations or changes to the sugar composition and/or level of glycosylation, that modulate Fc effector function.

In one embodiment, the term “Fc region” refers to the constant region of an antibody excluding the first constant region immunoglobulin domain. Thus, Fc refers to the last two constant region immunoglobulin domains of IgA. IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains CH2 and CH3 and the hinge between CH1 and CH. It is understood in the art that the boundaries of the Fc region may vary, however, the human IgG heavy chain Fc region is usually defined to comprise residues C226 or P230 to its carboxyl-terminus, using the numbering is according to the EU index as in Kabat et al. (1991, NIH Publication 91-3242, National Technical Information Service. Springfield, Va.). The term “Fc region” may refer to this region in isolation or this region in the context of an antibody or antibody fragment. “Fc region” includes naturally occurring allelic variants of the Fc region as well as modifications that modulate effector function. Fc regions also include variants that don't result in alterations to biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990).

In one embodiment, the term “antibody” refers to a protein functionally defined as a binding protein and structurally defined as comprising an amino acid sequence that is recognized by one of skill as being derived from the framework region of an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM. IgA. IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

In one embodiment, the term “antibody” includes antibody fragments that retain binding specificity. For example, there are a number of well characterized antibody fragments. Thus, for example, pepsin digests an antibody C-terminal to the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

Antibodies include VH-VL dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked VH-VL which may be expressed from a nucleic acid including VH- and VL-encoding sequences either joined directly or joined by a peptide-encoding linker (e.g., Huston, et al. Proc. Nat. Acad. Sci. USA. 85:5879-5883, 1988). While the VH and VL are connected to each as a single polypeptide chain, the VH and VL domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the invention also include heavy chain dimers, such as antibodies from camelids. In preferred embodiments, antibodies are employed in a form that can activate EphA3 present on the surface of pulmonary fibrotic cells and kill by ADCC. Thus, in some embodiments an antibody is dimeric. In other embodiments, the antibody may be in a monomeric form that has an active isotype. In some embodiments the antibody is in a multivalent form. e.g., a trivalent or tetravalent form, that can cross-link EphA3.

In one embodiment, the term “V-region” refers to an antibody variable region domain comprising the segments of Framework 1, CDR1, Framework 2. CDR2, and Framework3, including CDR3 and Framework 4, which segments are added to the V-segment as a consequence of rearrangement of the heavy chain and light chain V-region genes during B-cell differentiation.

In another embodiment, the term “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat. Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989. Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA. 86, 9268-9272 (1989); Martin, et al, Methods Enzymol., 203, 121-153. (1991); Pedersen et al, Immunomethods, 1, 126. (1992); and Rees et al, In Sternberg M. J. E. (ed.), Protein Structure Prediction. Oxford University Press, Oxford, 141-172 1996).

In one embodiment, the terms “Epitope” or “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

In one embodiment, the term “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci USA 91: 969, 1994).

In one embodiment, the term “human antibody” refers to an antibody that is substantially human. i.e., has FR regions, and often CDR regions, from a human immune system. Accordingly, the term includes humanized and HUMANEERED™ antibodies as well as antibodies isolated from mice reconstituted with a human immune system and antibodies isolated from display libraries.

In another embodiment, the term “humanized antibodies” refers a method of making an antibody having a binding specificity of a reference antibody, the method comprising: a) joining a heavy chain CDR3 binding specificity determinant (BSD) from the reference antibody to a diverse population of human NH segments thereby creating a library of human NH regions having the reference antibody heavy chain CDR3 BSD; b) joining a light chain CDR3 BSD from the reference antibody to a diverse population of human NL segments, thereby creating a library of human NL regions having the reference antibody light chain CDR3 BSD; c) combining the libraries of step a and step b to create an antibody library comprising members where a member has one NH comprising the reference antibody heavy chain CDR3 BSD and one NL comprising the reference antibody light chain CDR3 BSD; and d) isolating a member of the library of step c that binds the same antigen as the reference antibody. In one embodiment, the diverse population of human NH segments is human germline. The diverse population of human NL segments can also be human germline, or near human germline. Thus, both the diverse population of NH segment and the diverse population of NL segments can be human germline. In some embodiments, e.g., embodiments, in which a selected antibody has a germline NH and/or NL segment, the method further comprises mutagenizing one or both CDR3s from an antibody selected in step (d) and selecting an antibody that has a higher affinity for the target antigen than antibody selected in step (d).

In another embodiment, A “Humaneered™” (antibody in the context of this invention refers to an engineered human antibody having a binding specificity of a reference antibody. A “Humaneered™” antibody for use in this invention has an immunoglobulin molecule that contains minimal sequence derived from a donor immunoglobulin. Typically, an antibody is “Humaneered™” by joining a DNA sequence encoding a binding specificity determinant (BSD) from the CDR3 region of the heavy chain of the reference antibody to human VH segment sequence and a light chain CDR3 BSD from the reference antibody to a human VL segment sequence. Methods for humaneering are provided in US patent application publication no. 20050255552 and US patent application publication no. 20060134098, both of which are incorporated herein in their entirety.

In another embodiment, the term “hypofucosylated” antibody preparation refers to an antibody preparation in which the average content of α1,6-fucose is less than 50% of that found in naturally occurring IgG antibody preparations. As understood in the art, “hypofucosylated” is used in reference to a population of antibodies.

In another embodiment, the terms “non-fucosylated” or “afucosylated” antibody lacks α1,6-fucose attached to the CH2 domain of the IgG heavy chain.

In one embodiment, the term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid. Similarly, a heterologous protein will often refer to two or more subsequences that are not found in the same relationship to each other in nature.

In another embodiment, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus. e.g., recombinant cells express genes that are not found within the native (non-recombinant) form of the cell or express native genes that are otherwise abnormally expressed, under expressed or not expressed at all. In another embodiment, the term “recombinant nucleic acid” refers to a nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, and in another embodiment, a “recombinant protein” is a protein made using recombinant techniques. i.e., through the expression of a recombinant nucleic acid as depicted above.

In one embodiment, the phrase “specifically (or selectively) binds” to an antibody or “specifically (or selectively) immunoreactive with,” when referring to a protein or peptide, refers to a binding reaction where the antibody binds to the protein of interest. In the context of this invention, the antibody typically binds to EphA3 with an affinity that is at least 100-fold better than its affinity for other antigens.

In another embodiment, the term “equilibrium dissociation constant (KD) refers to the dissociation rate constant (kd, time−1) divided by the association rate constant (ka, time−1, M−1). Equilibrium dissociation constants can be measured using any known method in the art. The antibodies of the present invention are high affinity antibodies. Such antibodies have an affinity better than 500 nM, and often better than 50 nM or 10 nM. Thus, in some embodiments, the antibodies of the invention have an affinity in the range of 500 nM to 100 pM, or in the range of 25 nM to 100 pM, or in the range of 25 nM to 50 pM, or in the range of 25 nM to 1 pM.

In one embodiment, an “IPF therapeutic agent” refers to an agent that when administered to a patient suffering from IPF, in a therapeutically effective dose, will cure, or at least partially arrest the symptoms of the disease and complications associated with the disease.

In other embodiments, the terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

In one embodiment, the term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

In one embodiment, the term “Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid that encode a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C). Methionine (M) (see. e.g., Creighton. Proteins (1984)).

The term “a” or “an” is generally intended to mean “one or more” unless otherwise indicated.

### Description of Some Aspects of the Invention

The invention is based, in part, on the discovery that KB004 can be used to treat or prevent pulmonary fibrosis, e.g., treat IPF, when administered to a subject that has pulmonary fibrosis, e.g., IPF, or is at risk for having pulmonary fibrosis. Thus, in one aspect, the invention relates to a therapeutic antibody having the binding specificity of KB004 that is administered to a patient, e.g., a human patient that has a pulmonary fibrosis, such as IPF. A “patient” is most often a human patient. In some embodiments, the patient is a non-human mammal, e.g., a non-human primate, canine, equine, feline, bovine, ovine, and the like.

In some embodiments, the anti-EphA3 antibody comprises the CDRs of KB004. In some embodiments, the antibody comprises the V_{H} and V_{L} regions of KB004. In some embodiment, the antibody comprises heavy chain and light chains having the heavy and light chain sequence of KB004

In some embodiments, the term “binding specificity determinant” or “BSD” as used in the context of the current invention refers to the minimum contiguous or non-contiguous amino acid sequence within a CDR region necessary for determining the binding specificity of an antibody. In the current invention, the minimum binding specificity determinants reside within a portion or the full-length of the CDR3 sequences of the heavy and light chains of the antibody.

In some embodiments the therapeutic antibody has the binding specificity of KB004.

In some embodiments, the antibody is an anti-EphA3 antibody that compete with SL2, which binds to the extracellular domain of EphA3. SL2 is the monoclonal antibody, which is produced by a hybridoma deposited with the American Type Culture Collection (ATCC), 10801 University Blvd., Manassas. Va. 20110, USA on Nov. 8, 2011 under the conditions of the Budapest Treaty and assigned patent deposit number PTA-12227. In some embodiments, the antibody is a form of SL2 having substantially human framework sequences (e.g., at least 80% or 90% identity in the framework regions to human germline framework regions.

In some embodiments, an anti-EphA3 antibody for treating pulmonary fibrosis, e.g., IPF, does not activate EphA3. In some embodiments, the anti-EphA3 antibody blocks binding of an ephrin ligand to EphA3. In some embodiments, the anti-EphA3 antibody has a high affinity and does not block binding to EphA3. In some embodiments, the anti-EphA3 antibody is conjugated to a therapeutic agent, such as a cytotoxic moiety. In some embodiments, the anti-EphA3 antibody clusters EphA3 on the cell surface sufficient to activate EphA3.

In some embodiments the anti-EphA3 antibodies are humanized monoclonal antibodies or engineered to contain framework sequences that are close to germline (see, e.g., U.S. Pat. No. 8,664,365). As noted supra, humanized forms of antibodies are chimeric immunoglobulins in which a CDR of a human antibody is replaced by a CDR of a non-human species such as mouse, rat or rabbit having the desired specificity, affinity and capacity.

An antibody that is employed in the invention can be in numerous formats. In some embodiments, the antibody can include an Fc region, e.g., a human Fc region. For example, such antibodies include IgG antibodies that bind EphA3 and that have an active isotype. In some embodiments, the antibody can be an active fragment (e.g., it can dimerize EphA3) or can comprise a derivative of an antibody such as an Fab. Fab′. F(ab′)₂. Fv, scFv, or a single domain antibody (“dAb”). For example, in some embodiments, the antibody may be a F(ab′)2. Other exemplary embodiments of antibodies that can be employed in the invention include activating nanobodies or activating camellid antibodies. Such antibodies may additionally be recombinantly engineered by methods well known to persons of skill in the art. As noted above, such antibodies can be produced using known techniques. As appreciated by one of skill in the art, in some embodiments when an antibody is in a format that can be monovalent, e.g., an Fv or Fab format, the antibody may be employed as a multivalent antibody, such as a trivalent or tetravalent antibody. Methods of generating multivalent antibodies are known (see, e.g., King et al., Cancer Res. 54:6176-6185, 1994).

An antibody for use in the invention typically has an Fc constant region that has an effector function, e.g., binds to an Fc receptor present on immune effector cells. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g., B cell receptor), and the like. Such effector functions generally require the Fc region to be combined with a binding domain (e.g. an antibody variable domain) and can be assessed using known assays (see. e.g., the references cited hereinbelow.)

Anti-EphA3 antibodies that have an active isotype and are bound to Fc-receptors on effector cells, such as macrophages, monocytes, neutrophils and NK cells, can induce cell death by ADCC.

The Fc region can be from a naturally occurring IgG1, or other active isotypes, including IgG3, IgM, IgA, and IgE. “Active isotypes” include antibodies where the Fc region comprises modifications to increase binding to the Fc receptor or otherwise improve the potency of the antibody. Such an Fc constant region may comprise modifications, such as mutations, changes to the level of glycosylation and the like, that increase binding to the Fc receptor. There are many methods of modifying Fc regions that are known in the art. For example, U.S. Patent Application Publication No. 20060039904 describes variants of Fc receptors that have enhanced effector function, including modified binding affinity to one or more Fc ligands (e.g., FcyR, C1q). Additionally, such Fc variants have altered antibody-dependent cell-mediated cytotoxicity (ADCC) and/or complement dependent cytotoxicity (CDC) activity. Other Fc variants include those disclosed by Ghetie et al., Nat Biotech. 15:637-40, 1997; Duncan et al. Nature 332:563-564, 1988; Lund et al., J. Immunol 147:2657-2662, 1991; Lund et al, Mol Immunol 29:53-59, 1992; Alegre et al. Transplantation 57:1537-1543, 1994; Hutchins et al., Proc Natl. Acad Sci USA 92:11980-11984, 1995; Jefferis et al, Immunol Lett. 44:111-117, 1995; Lund et al., FASEB J 9:115-119, 1995; Jefferis et al. Immunol Lett 54:101-104, 1996; Lund et al. J Immunol 157:4963-4969, 1996; Armour et al., Eur J Immunol 29:2613-2624, 1999; Idusogie et al. J Immunol 164:4178-4184, 200; Reddy et al, J Immunol 164:1925-1933, 2000; Xu et al., Cell Immunol 200:16-26, 2000; Idusogie et al. J Immunol 166:2571-2575, 2001; Shields et al., J Biol Chem 276:6591-6604, 2001; Jefferis et al, Immunol Lett 82:57-65, 2002; Presta et al., Biochem Soc Trans 30:487-490, 2002; Lazar et al., Proc. Natl. Acad. Sci. USA 103:4005-4010, 2006; U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7,335,742; and 7,317,091; and PCT Publications WO 94/2935; WO 99/58572; WO 00/42072; WO 02/060919, and WO 04/029207.

In some embodiments, the natural glycosylation of Fc regions may be modified. For example, a modification may be aglycosylation, for example, by removing one or more sites of glycosylation within the antibody sequence. Such an approach is described in further detail in U.S. Pat. Nos. 5,714,350 and 6,350,861. An Fc region can also be made that has an altered type of glycosylation, such as a hypofucosylated Fc variant having reduced amounts of fucosyl residues or an Fc variant having increased bisecting GlcNAc structures. Such carbohydrate modifications can be accomplished by, for example, expressing the antibody in a host cell with altered glycosylation machinery. Cells with altered glycosylation pathways, including mammalian cells, yeast and plants, have been described in the art and can be used as host cells in which to express recombinant antibodies of the invention to thereby produce an antibody with altered glycosylation. Techniques for modifying glycosylation include those disclosed e.g., in Umana et al. Nat. Biotechnol 17:176-180, 1999; Davies, et al., Biotechnol. Bioeng. 74:288-294, 2001; Shields et al, J Biol Chem 277:26733-26740, 2002; Shinkawa et al., J Biol Chem 278:3466-3473, 2003; Niwa et al. Clinc. Cancer Res. 1-:6248-6255, 2004; Presta et al., Biochem Soc Trans 30:487-490, 2002; Kanda et al, Glycobiology 17:104-118, 2006; U.S. Pat. Nos. 6,602,684; 6,946,292; and 7,214,775; U.S. Patent Application Publication Nos. 20070248600; 20070178551; 20080060092; 20060253928; PCT publications WO 00/61739; WO 01/292246; WO 02/311140; and WO 02/30954; and Potelligent™ technology (Biowa. Inc. Princeton, N.J.); and GlycoMAb™ glycosylation engineering technology (GLYCART biotechnology AG. Zurich, Switzerland). Non-glycosylated antibodies may also be produced using ProBioGen technology (von Horsten et al., Glycobiology 20(12): 1607-1618, 2010). In a hypofucosylated antibody preparation, typically at least 50 to 70% of the antibody molecule, often at least 80% of the molecules, or at least 90% of the molecules, lack fucose.

In some embodiments of the invention, the antibody is additionally engineered to reduce immunogenicity, e.g., so that the antibody is suitable for repeat administration. Methods for generating antibodies with reduced immunogenicity include humanization and humaneering procedures and modification techniques such as de-immunization, in which an antibody is further engineered, e.g., in one or more framework regions, to remove T cell epitopes.

In some embodiments, the anti-EphA3 antibody of the present invention is a HUMANEERED™ anti-EphA3 antibody or a chimeric anti-EphA3 antibody.

In some embodiments, the variable region is comprised of human V-gene sequences. For example, a variable region sequence can have at least 80% identity, or at least 85% or at least 90% identity, or higher, to human germ-line V-gene sequences.

In another embodiment, the invention includes a homologue of an anti-EphA3 antibody. In another embodiment, the invention includes a homologue of KB004. In another embodiment, the invention includes a homologue of an anti-EphA3 antibody have the binding specificity of KB004. In another embodiment, the invention employs the use of homologues e.g., polypeptides which are at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homologous to KB004 as determined using BlastP software of the National Center of Biotechnology Information (NCBI) using default parameters.

In some embodiments, the variable region sequence of the anti-EphA3 antibody have at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homology to the heavy chain variable sequence or light chain variable sequence of KB004. In some embodiments, the heavy region sequence of the anti-EphA3 antibody have at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homology to the heavy chain sequence or kappa chain sequence or any subsequences (e.g. CDR, constant region, framework refion) of KB004. In some embodiments, the signal sequence of the anti-EphA3 antibody have at least 50%, at least 55%, at least 60%, at least 65%, at least 70%, at least 75%, at least 80%, at least 85%, at least 87%, at least 89%, at least 91%, at least 93%, at least 95%, at least 96%, at least 97%, at least 98%, or at least 99% homology to the heavy chain signal sequence or light chain signal sequence of KB004.

For expression in some cell systems, a signal peptide can be introduced at the N-terminus to direct secretion to the extracellular medium. Antibodies may be secreted from bacterial cells such as E. coli with or without a signal peptide. In some embodiments, the signal peptide of the anti-EphA3 antibody is cleaved once the mature anti-EphA3 antibody is expressed.

An antibody used in the invention can include a human constant region. The constant region of the light chain may be a human kappa or lambda constant region. The heavy chain constant region is often a gamma chain constant region, for example, a gamma-1 or gamma-3 constant region.

In some embodiments, e.g., where the antibody is a fragment, the antibody can be conjugated to another molecule, e.g., to provide an extended half-life in vivo such as a polyethylene glycol (pegylation) or serum albumin. Examples of PEGylation of antibody fragments are provided in Knight et al., Platelets 15:409, 2004 (for abciximab); Pedley et al., Br. J. Cancer 70:1126, 1994 (for an anti-CEA antibody); and Chapman et al., Nature Biotech. 17:780, 1999.

### Antibody Specificity

An antibody for use in the methods of the invention activates EphA3 and/or kills EphA3+ cells by ADCC. In some embodiments, the antibody activates Epha3 and induces ADCC. An example of an antibody suitable for use with the present invention is an antibody that has the binding specificity of KB004 or mAb IIA4, which IIIA4 binds to the native EphA3 globular ephrin-binding domain (Smith et al., J. Biol. Chem. 279:9522-9531, 2004; and Vearing et al., Cancer Res. 65:6745-6754, 2005). High affinity mAb IIIA4 binding to the EphA3 surface has little effect on the overall affinity of ephrin-A5 interactions with EphA3.

In some embodiments, a monoclonal antibody that competes with KB004 for binding to EphA3, or that binds the same epitope as KB004, is used. Any of a number of competitive binding assays can be used to measure competition between two antibodies for binding to the same antigen. For example, a sandwich ELISA assay can be used for this purpose. In an exemplary assay. ELISA is carried out by using a capture antibody to coat the surface of a well. A subsaturating concentration of tagged-antigen is then added to the capture surface. This protein will be bound to the antibody through a specific antibody:antigen interaction. After washing, a second antibody that is linked to a detectable moiety is added to the ELISA. If this antibody binds to the same site on the antigen as the capture antibody, or interferes with binding to that site, it will be unable to bind to the target protein as that site will no longer be available for binding. If, however, this second antibody recognizes a different site on the antigen it will be able to bind. Binding can be detected by quantifying the amount of detectable label that is bound. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine specificity. The ability of a particular antibody to recognize the same epitope as another antibody is typically determined by such competition assays.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

In some embodiments, the antibody binds to the same epitope as mAb IIIA4. The epitope for mAb IIIA4 and human engineered derivatives resides in the N-terminal globular ligand binding domain of EphA3 (amino acids 29-202 in the partial human EphA3 sequence below):

| (SEQ ID NO: 1) | |
|---|---|
| 1 | MDCQLSILLL LSCSVLDSFG ELIPQPSNEV NLLDSKTIQG ELGWISYPSH GWEEISGVDE |
| 61 | HYTPIRTYQV CNVMDHSQNN WLRTNWVPRN SAQKIYVELK FTLRDCNSIP LVLGTCKETF |
| 121 | NLYYMESDDD HGVKFREHQF TKIDTIAADE SFTQMDLGDR ILKLNTEIRE VGPVNKKGFY |
| 181 | LAFQDVGACV ALVSVRVYFK KC |

The mAb IIIA4 antibody binds adjacent to but does not interfere substantially with binding of EphrinA5 to the receptor. The epitope for mAb IIIA4 has been further characterized by Smith et al., J. Biol. Chem. 279: 9522, 2004 using site-directed mutagenesis. In this analysis, mutation of Glycine at position 132 to Glutamic acid (G132E) abolishes binding to mAb IIIA4. Mutation of Valine 133 to Glutamic acid (V133E) reduces binding of EphA3 to mAb IIIA4 antibody approximately 100-fold. It has subsequently been observed by the inventors that Arginine 136 is also part of the epitope. This residue is changed to Leucine in the sequence of the highly conserved EphA3 protein in the rat (R136L). Rat EphA3 does not bind mAb IIIA4 or a human engineered derivative of mAb IIIA4. Thus, G132, V133 and R136 (bolded and underlined in the sequence above) are important amino acids within the mab IIIA4 epitope.

### Binding Affinity

Anti-EphA3 antibodies suitable for use with the present invention typically have a high affinity binding for human EphA3. For the purposes of this invention, high affinity binding between an antibody and an antigen exists if the dissociation constant (K_{D}) of the antibody is < about 10 nM, for example, about 5 nM, or about 2 n M, or about 1 nM, or less. A variety of methods can be used to determine the binding affinity of an antibody for its target antigen such as surface plasmon resonance assays, saturation assays, or immunoassays such as ELISA or RIA, as are well known to persons of skill in the art. An exemplary method for determining binding affinity is by surface plasmon resonance analysis on a BIAcore 2000 instrument (Biacore AB, Freiburg, Germany) using CM5 sensor chips, as described by Krinner et al., (2007) Mol. Immunol. February; 44(5):916-25. (Epub 2006 May 11)).

In one embodiment, a therapeutic antibody is an anti-EphA3 antibody disclosed herein.

The therapeutic anti-EphA3 antibody anti-EphA3 antibody activates EphA3. Often, the antibody multimerizes, e.g., dimerizes. EphA3. In some embodiments, the antibody clusters EphA3. In some embodiments, an anti-EphA3 antibody can also be employed that has an active isotype, such as an IgG1, IgG3, IgM, IgA, or IgE, and is cytotoxic to fibrotic cells via ADCC. Antibodies for use in the invention can also be multivalent including forms of monomers that are cross-linked or otherwise multimerized to form multivalent antibodies.

Antibodies for use in the methods and compositions described herein can be identified using known assays for the characteristic of interest. Thus, antibodies can be identified by screening for the ability to activate EphA3 (e.g., using a phosphorylation assay and/or screening for changes in cell morphology, such as rounding), the ability to induce ADCC, and for binding specificity and affinity. Such assays are well known in the art.

### Diagnostic Antibodies

Therapeutic antibodies may also be used for diagnosis of IPF to identify an IPF patient that is a candidate for treatment with KB004 or an antibody having the binding specificity of KB004. Alternatively, other anti-EphA3 antibodies may be used, so long as the anti-EphA3 antibody binds to EphA3, e.g., the extracellular domain of EphA3.

### Treatment of IPF

In one aspect, the methods of the present invention comprise administering an anti-EphA3 antibody having the binding specificity of KB004, or administering KB004, to a patient that has IPF where fibrotic cells express EphA3. Various methods, such as immunohistochemistry and/or PCR and the like can be employed to determine patients have have fibrotic cells that express EphA3.

The anti-EphA3 antibody, e.g., KB004 or a monoclonal antibody having the binding specificity of KB004, for administering to the patient can be formulated for use in a variety of drug delivery systems. One or more physiologically acceptable excipients or carriers can also be included in the compositions for proper formulation. Suitable formulations for use in the present invention are found in Remington: The Science and Practice of Pharmacy, 22nd Edition, 2012 Philadelphia, Pa. Lippincott Williams & Wilkins, 2005.

The anti-EphA3 antibody for use in the methods of the invention is provided in a solution suitable for injection into the patient such as a sterile isotonic aqueous solution for injection. The anti-EphA3 antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

The pharmaceutical compositions of the invention are administered to a patient that has IPF in an amount sufficient to at least partially arrest the disease or symptoms of the disease and its complications. In one embodiment, an amount adequate to accomplish this is defined as a “therapeutically effective dose.” A therapeutically effective dose is determined by monitoring a patient's response to therapy. Typical benchmarks indicative of a therapeutically effective dose are known in the art, depending on the disease. For example, therapeutic efficacy may be indicated by clinical symptoms such as improved breathing, decreased hallmarks of fibrosis or pneumonia, or other clinical assays.

The dose of the anti-EphA3 antibody is chosen in order to provide effective therapy for the patient and is in the range of about 0.1 mg/kg body weight to about 10 mg/kg body weight or in the range about 1 mg to about 1 g per patient. The dose is often in the range of about 0.5 mg/kg or about 1 mg/kg to about 10 mg/kg, or approximately about 50 mg to about 1000 mg/patient. In some embodiments, the antibody is administered in an amount less than about 0.1 mg/kg body weight. e.g., in an amount of about 20 mg/patient or less. The dose may be repeated at an appropriate frequency which may be in the range once per day to once every three months, depending on the pharmacokinetics of the antibody (e.g. half-life of the antibody in the circulation) and the pharmacodynamic response (e.g. the duration of the therapeutic effect of the antibody). In some embodiments where the antibody or modified antibody fragment has an in vivo half-life of between about 7 and about 25 days and antibody dosing is repeated between once per week and once every 3 months. In other embodiments, the antibody is administered approximately once per month.

Amounts that are administered that are effective will depend upon the severity of the disease and the general state of the patient's health, including other factors such as age, weight, gender, administration mute, etc. Single or multiple administrations of the anti EphA3 antibody may be administered depending on the dosage and frequency as required and tolerated by the patient. In any event, the methods provide a sufficient quantity of the anti EphA3 antibody to effectively treat the pulmonary fibrosis. e.g., IPF.

An anti-EphA3 antibody may be used with another therapeutic agent for the treatment of IPF. Patients can receive one or more of these additional therapeutic agents as concomitant therapy. Alternatively, patients may be treated sequentially with additional therapeutic agents.

In some embodiments. KB004, or an antibody having the binding specificity of KB004, is administered by injection or infusion through any suitable route, typically intravenous routes. In some embodiments, the anti-EphA3 antibody is diluted in a physiological saline solution for injection prior to administration to the patient. The antibody is administered, for example, by intravenous infusion over a period of between 15 minutes and 2 hours.

The following examples are provided by way of illustration only and not by way of limitation. Those of skill in the art will readily recognize a variety of non-critical parameters that could be changed or modified to yield essentially similar results.

### EXAMPLES

### Example 1: EphA3 in Idiopathic Pulmonary Fibrosis and Cancer

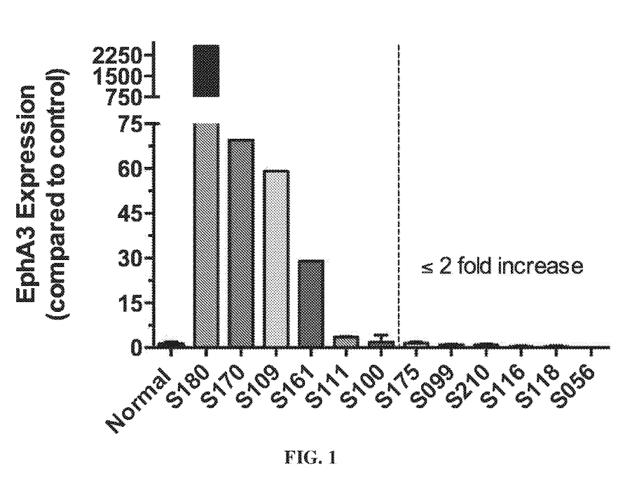

EphA3 transcript expression was observed in primary human fibroblasts cultures from IPF patients (FIG. 1).

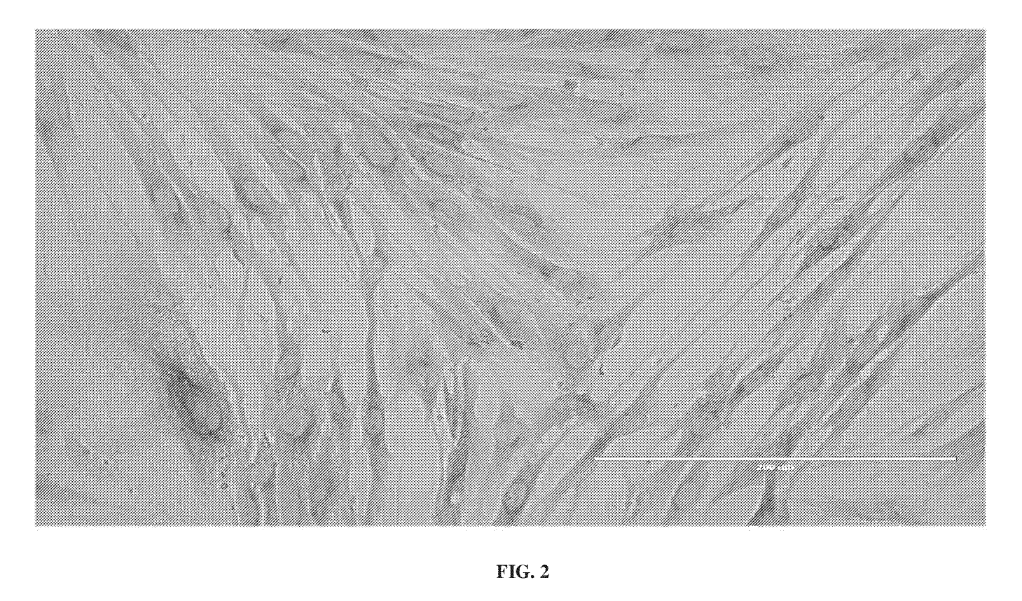

FIG. 2 provides data showing that EphA3 was expressed in fibrotic lung in IPF.

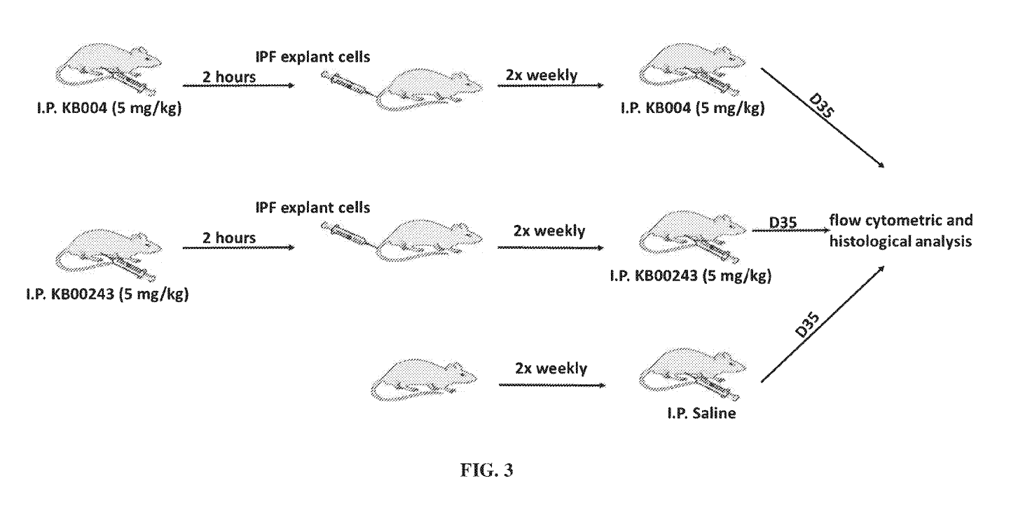

FIG. 3 shows the experiments design to evaluate the efficacy of KB004 in treating IPF in a SCID/NOD mouse model of IPF (see. e.g., B. Moore, Bethany, et al. “Animal models of fibrotic lung disease.” Am J Respir Cell Mol Biol. 2013 August; 49(2): 167-179).

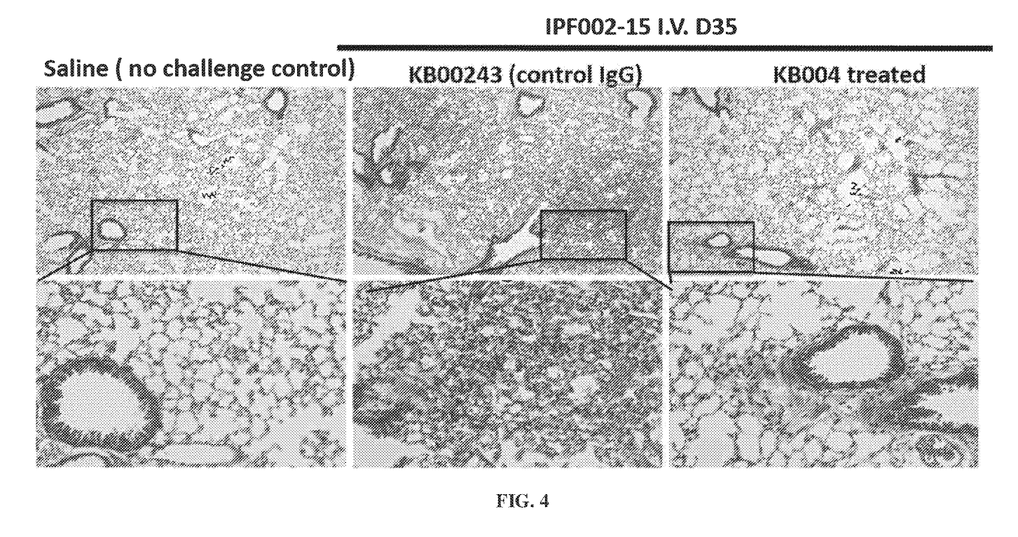

FIG. 4 provides data illustrating that KB004 treatment resulted in reduced lung fibrosis compared to control immunoglobulin G (IgG) (KB00243). Histological analysis was performed by trichrome staining at day 35.

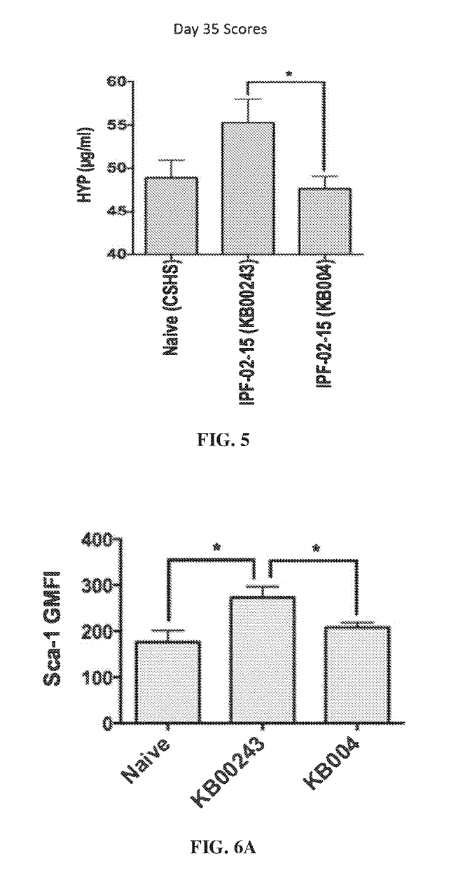

FIG. 5 provides data illustrating that hydroxyproline (fibrosis) levels in the lungs of KB004 vs. control IgG-treated mice were reduced.

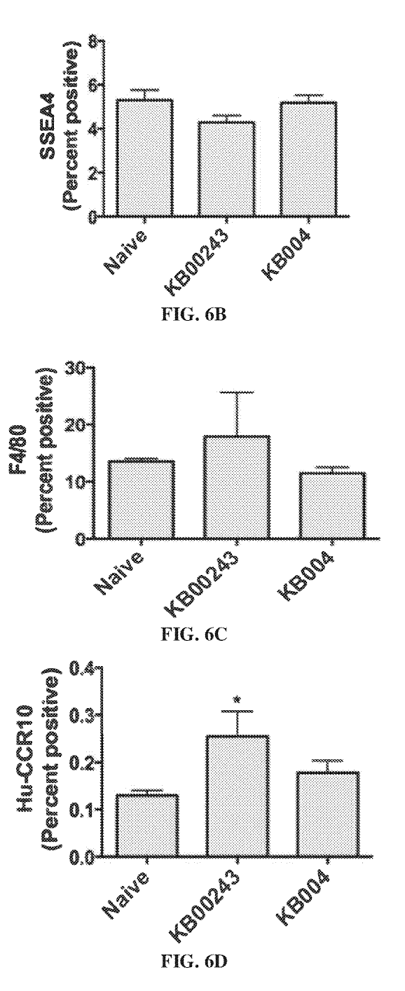

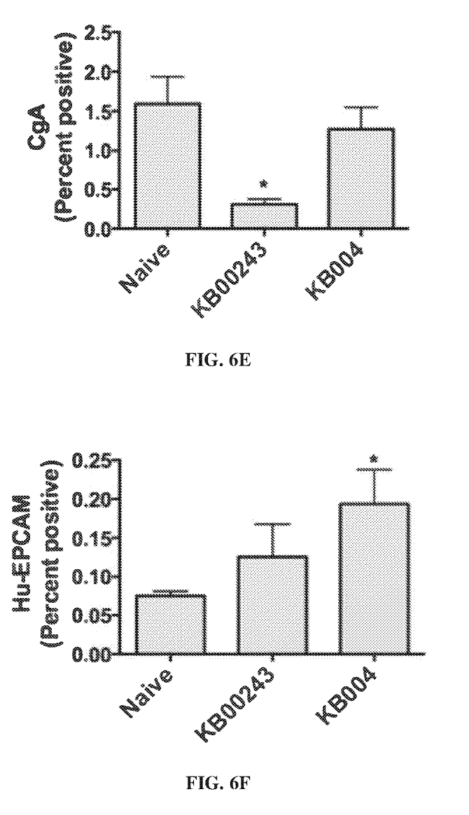

FIG. 6 provides data illustrating that disease-associate markers were reduced in cells in lungs of KB004-treated vs. control-treated mice.

An anti-EphA3 antibody SL2 that binds to the extracellular domain of EphA3 was also administered to mice in the SCID/NOD mouse model of IPF. The results show that SL2 also exhibited a therapeutic benefit.

The experiments provided herein thus show that EphA3 expression is detected on ‘diseased’ fibroblasts from IPF subjects and further, showed that in a SCID/NOD mouse model of IPF, treatment with KB004 reduced disease markers of fibrosis; and that KB004 reduces lung fibrosis at day 35 (histological analysis).

### Example 2: Expression and Targeting of EphA3 in IPF

Expression of EphA3 in IPF Lung

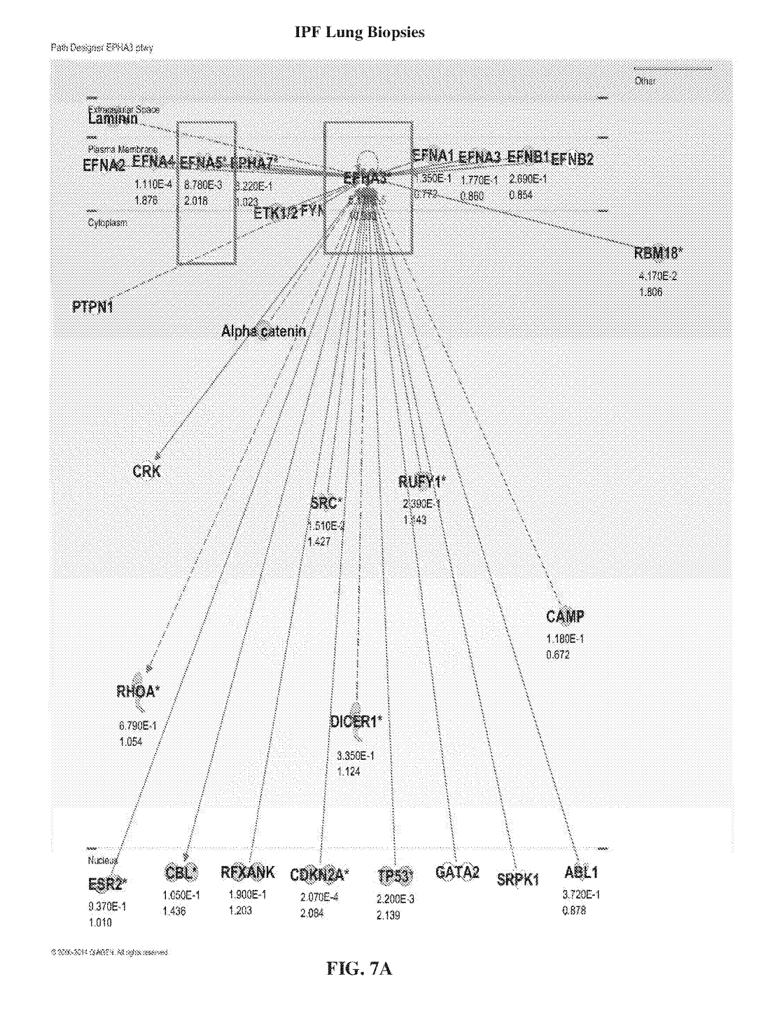

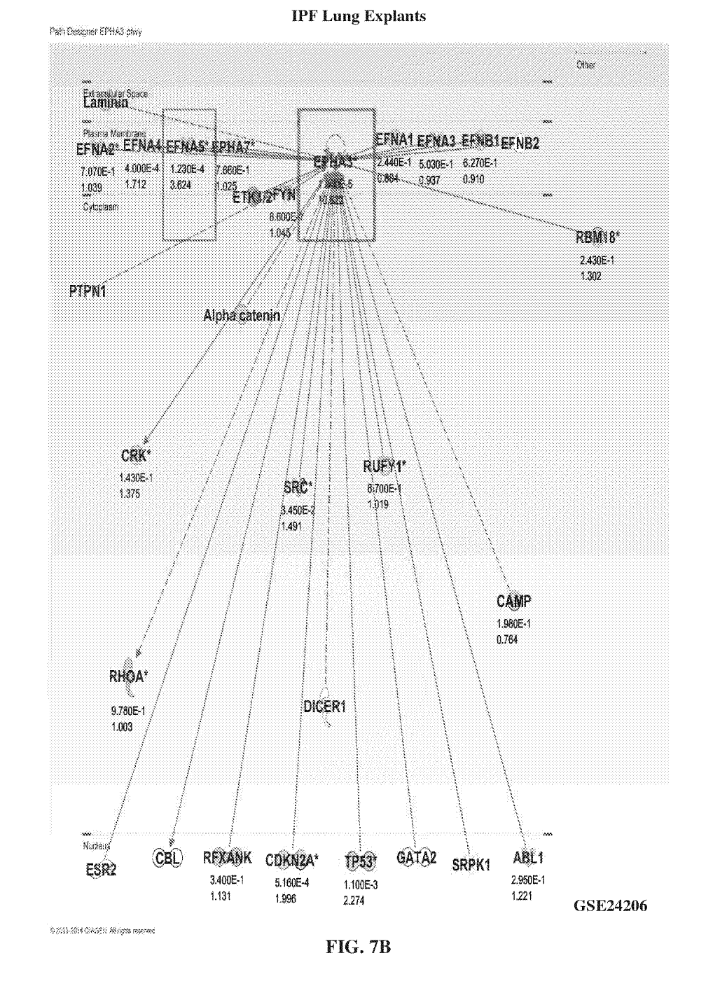

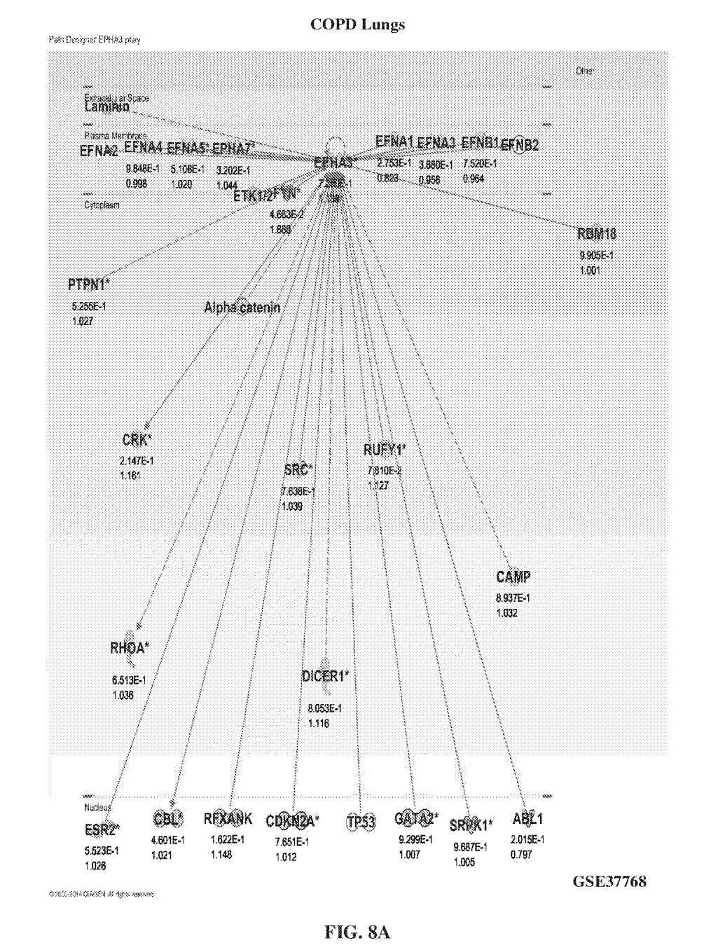

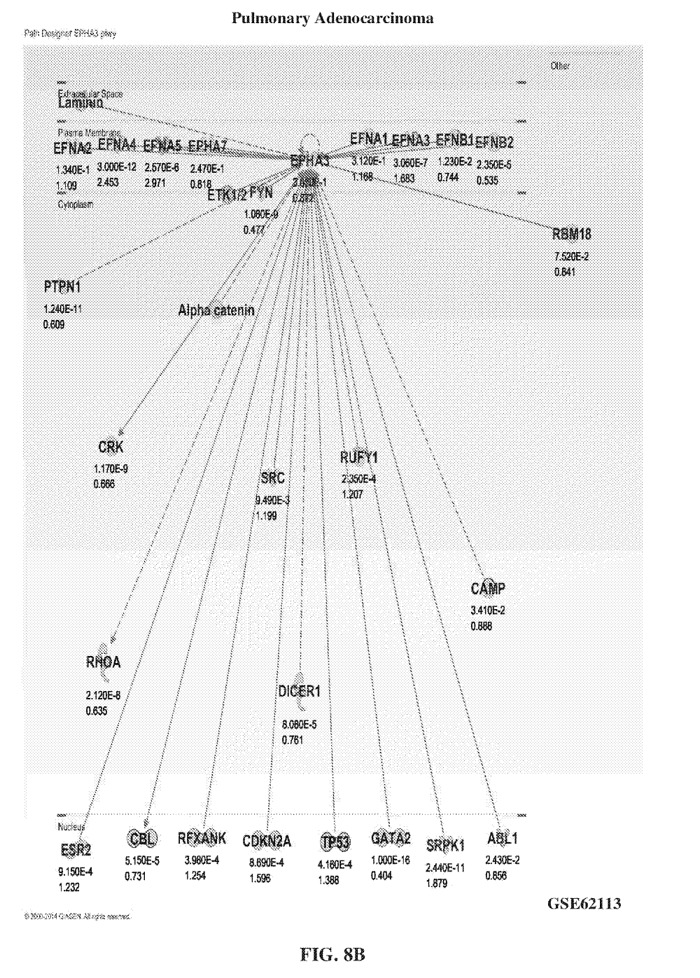

IPF lung biopsies and IPF lung explants showed a higher expression of EphA3 compared to lung samples taken from other lung diseases, such as chronic obstructive pulmonary disease (COPD) or pulmonary adenocarcinoma. FIG. 7A-FIG. 7B showed the high levels of EphA3 in IPF lung biopsies (FIG. 7A) and IPF lung explants (FIG. 7B). FIG. 8A-FIG. 8B showed the levels of EphA3 in COPD lungs (FIG. 8A) and pulmonary adenocarcinoma (FIG. 8B).

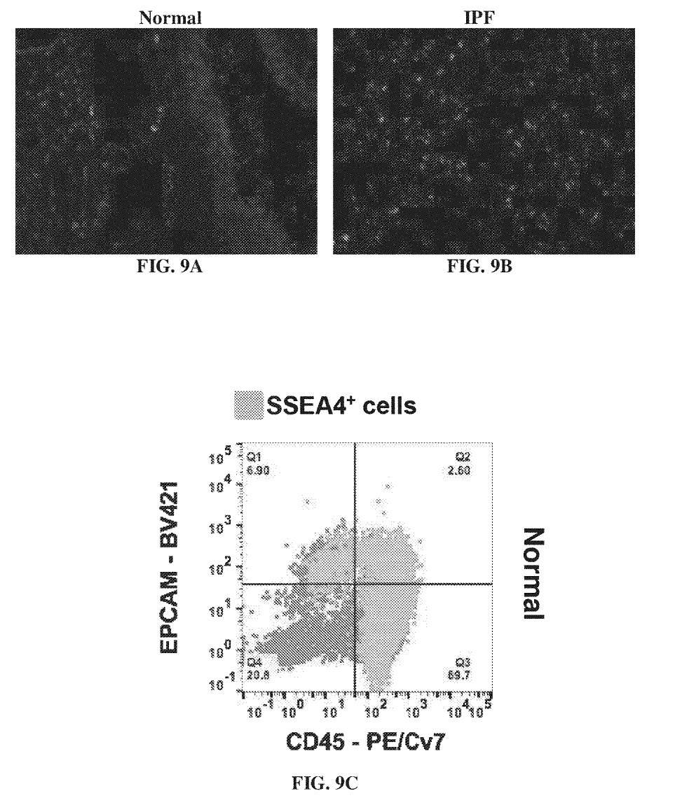

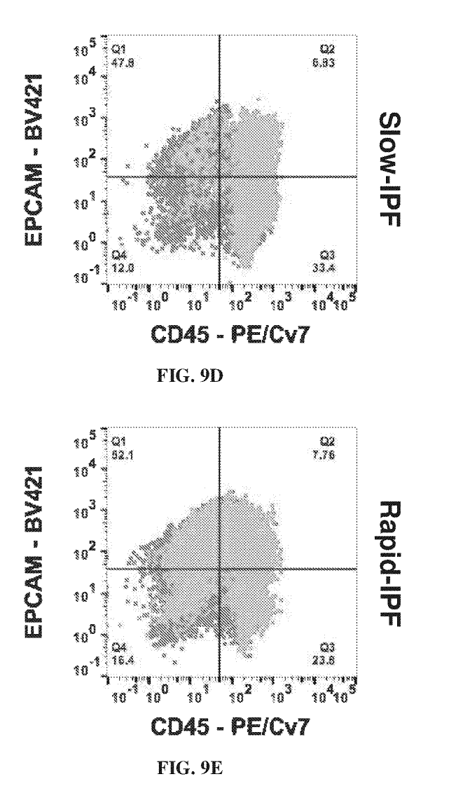

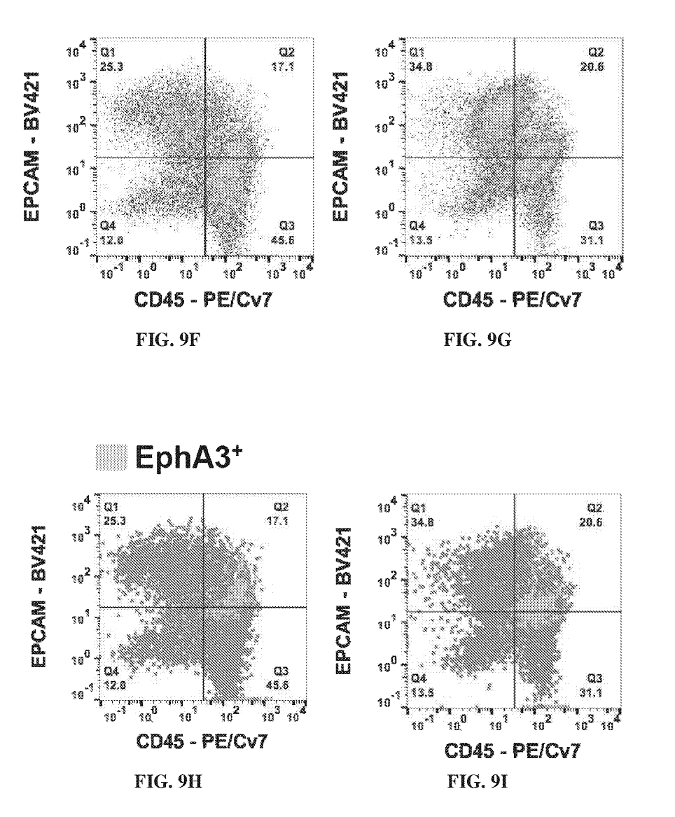

SSEA4+ stromal cell progenitors in IPF were EphA3 positive. FIG. 9A-FIG. 9I provided hematologic data illustrating that stage-specific embryonic antigen 4 (SSEA-4+) stromal cell progenitors in an IPF lung are EphA3 positive compared to normal lungs. FIG. 9A showed the fluorescent staining of SSEA-4+ stromal cell progenitors in a normal lung. FIG. 9B showed the fluorescent staining of SSEA-4+ stromal cell progenitors in an idiopathic pulmonary fibrosis (IPF) lung. Scatterplots of the detection of CD45 and EpCAM markers in SSEA-4+ cells were shown for normal lungs (FIG. 9C), slow progression IPF (FIG. 9D), and rapid progression IPF (FIG. 9E). The data illustrated in FIG. 9F-FIG. 9I showed the stromal progenitor cells CD45+ and EpCAM express EphA3.

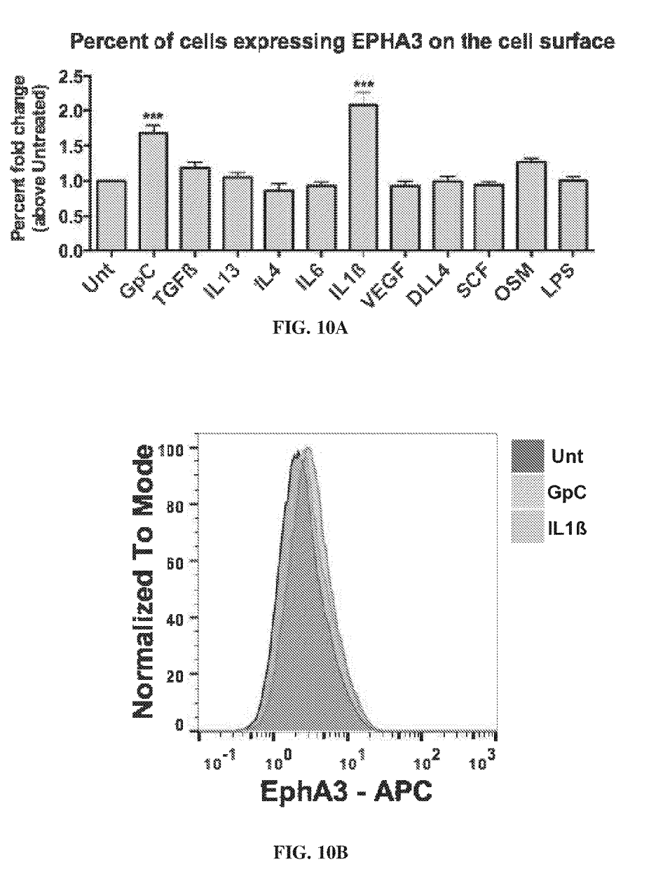

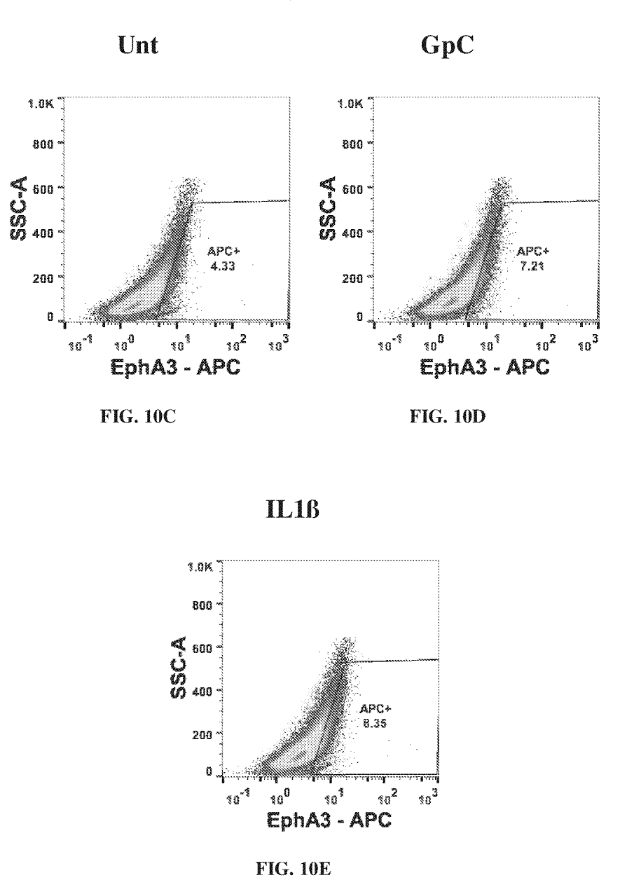

FIG. 10A-FIG. 10E show the regulation of EphA3 in fibroblast/progenitor cultures. FIG. 10A provides data showing the percent fold change, above untreated, of cells expressing EphA3 on the cell surface. FIG. 10B. provides flow cytometry data showing the concentrations of EphA3 normalized to mode for untreated (Unt), GpC, and interleukin 1 beta (IL1β) cells. FIG. 10C-FIG. 10E show the side scatter light flow cytometry plots for untreated (FIG. 10C), GpC (FIG. 10D), and IL1 IL1β (FIG. 10E).

Role of Epha3 in Freshly Isolated IPF Lung Explant Cellular Suspensions

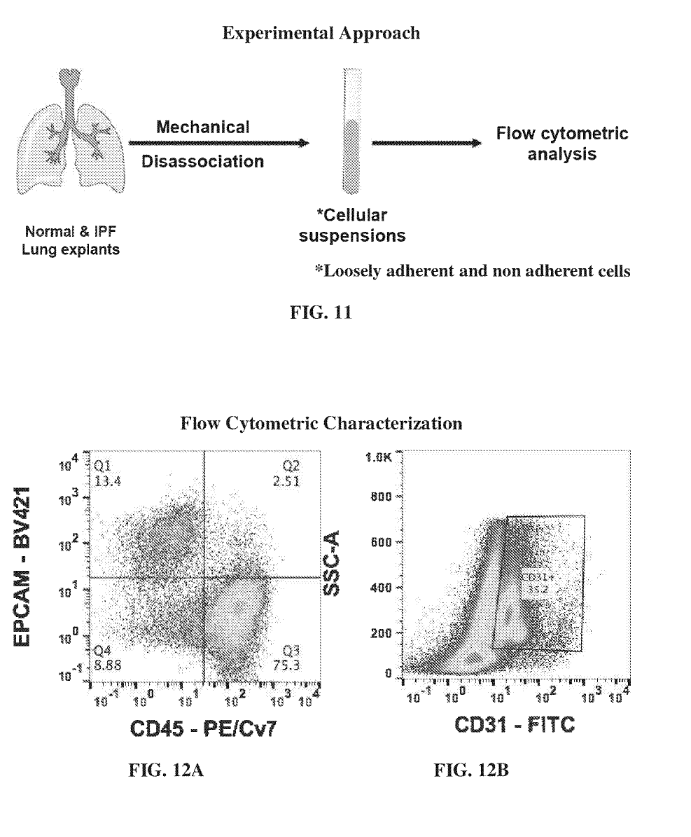

An experiment testing the role of Epha3 in isolated IPF lung explants was conducted. The experimental design is shown in FIG. 11. Normal and IPF lung explants were mechanically disassociated and held in cellular suspensions. The cellular suspension included loosely adherent and non-adherent cells. Flow cytometric analyses were performed on these cellular suspensions.

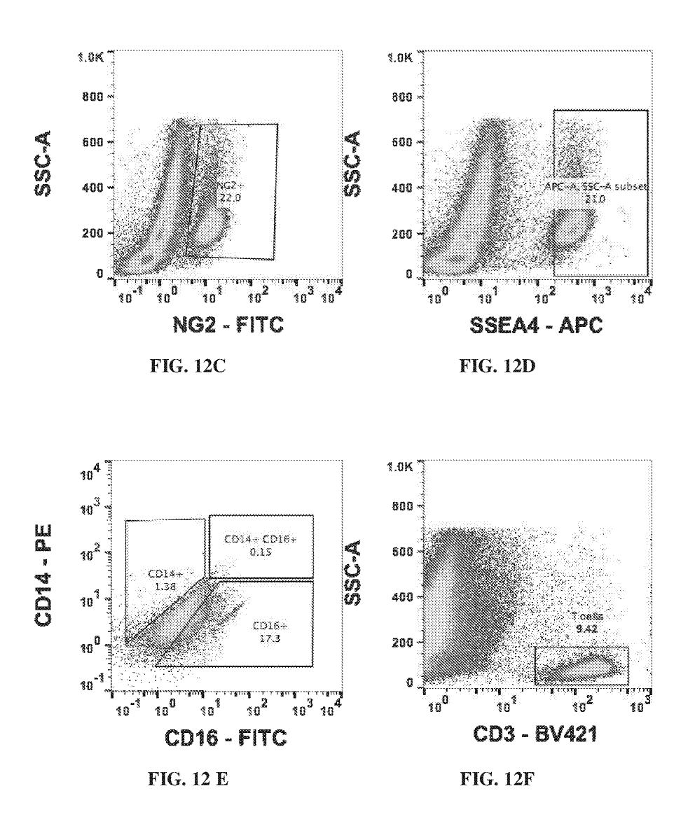

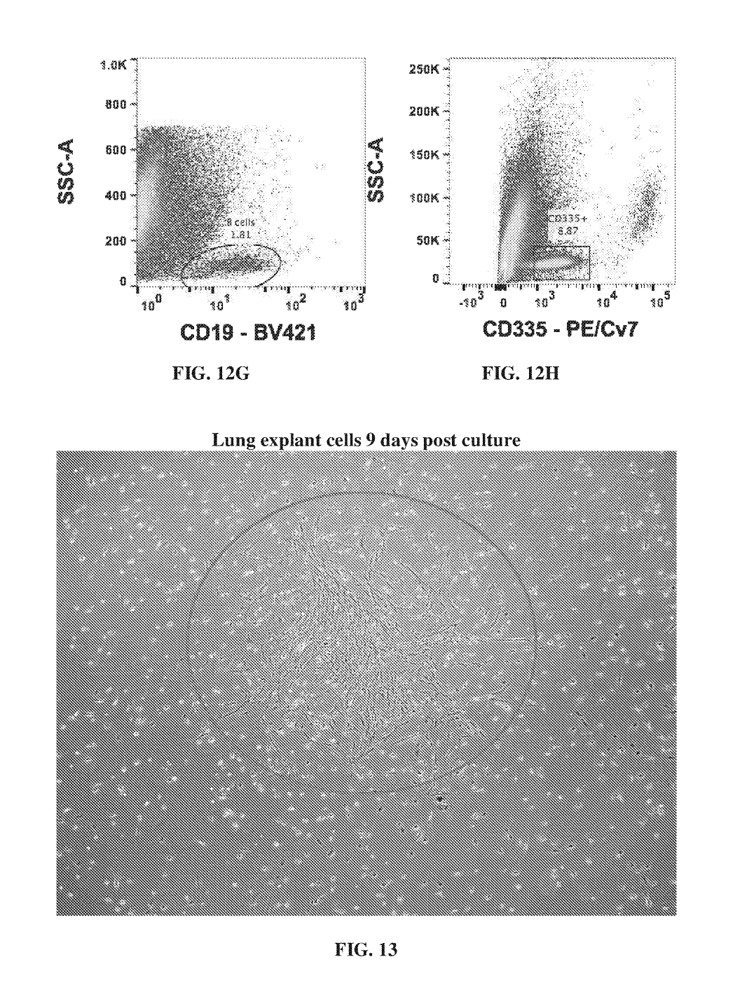

Flow cytometry showed the high expression of CD45 versus EPCAM in the cellular suspension of IPF lung explants (FIG. 12A). The flow cytometry data for the expression of SSEA-4 in the cellular suspensions is shown in FIG. 12D.

Fibroblast precursors were observed in lung explant cellular suspension 9 days post culture (FIG. 13).

KB004 Treatment in Fresh IPF Cells Taken from Explanted Lung

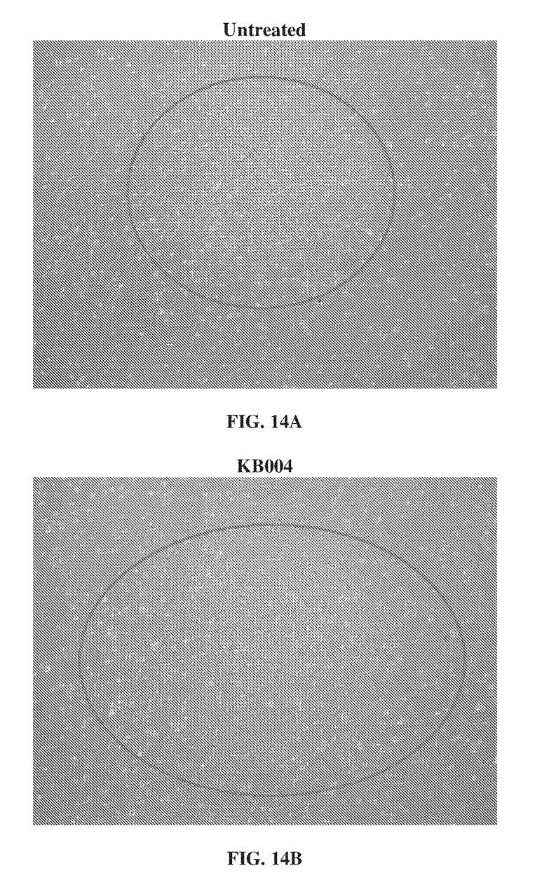

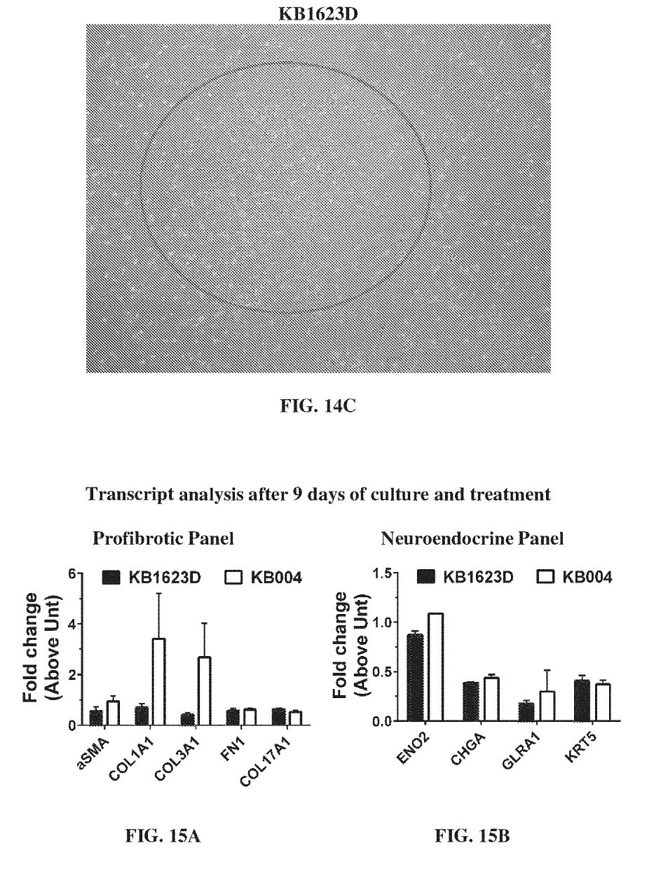

Cellular suspension of the IPF lung explants were treated with 20 μg/ml of an antibody, either KB004 or KB1623D, once every three days. One cellular suspension was left untreated. The appearance of any fibroblast colonies was observed 9 days post culture for the untreated (FIG. 14A), KB004 (FIG. 14B), and KB1623D (FIG. 14C) samples.

Transcript analysis after 9 days of culture and treatment was performed. The profibrotic panel is shown in FIG. 15A and the neuroendocrine panel is shown in FIG. 15B.

The activation of EphA3 leads to expansion of fibroblasts from fibroblast progenitors in an in vitro system in which immune cells are absent (due to non-adherence). Another antibody (SL2) that neutralizes EphA3 inhibits fibroblast expansion.

Modeling IPF in NOD/SCID Mice

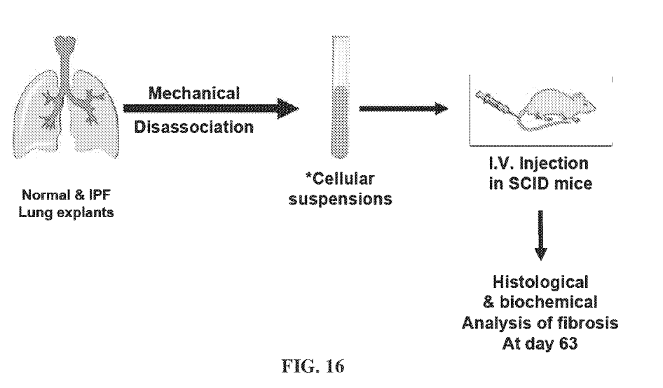

An experiment modeling IPF in NOD/SCID mice was performed and is outlined in FIG. 16. Normal and IPF lung explants were mechanically disassociated and held in cellular suspensions. The cellular suspension included loosely adherent and non-adherent cells. The cellular suspensions were then intravenously injected into SCID mice. Histological and biochemical analyses of fibrosis were performed 63 days after injection.

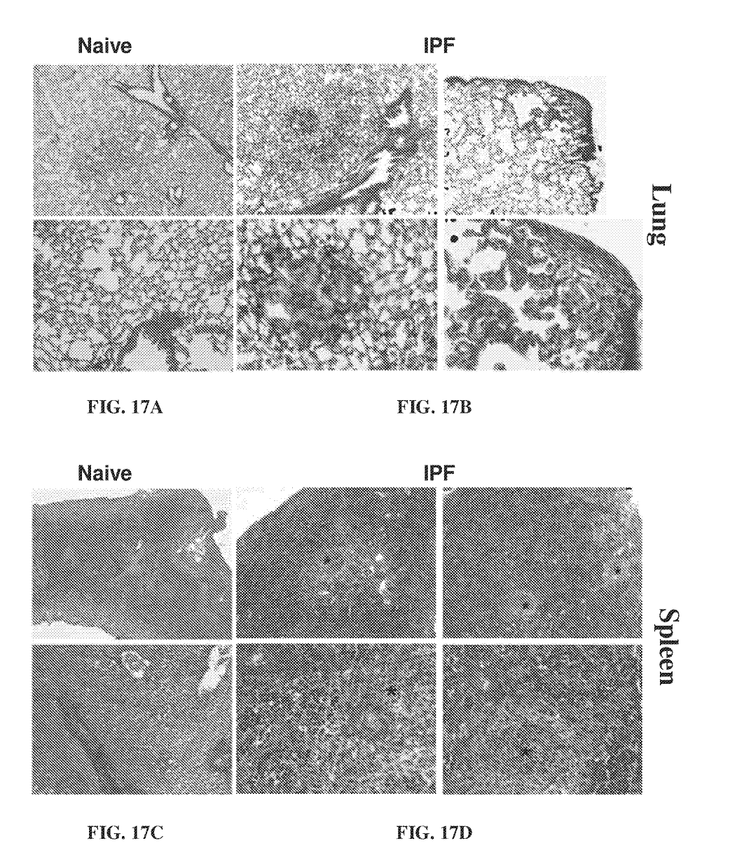

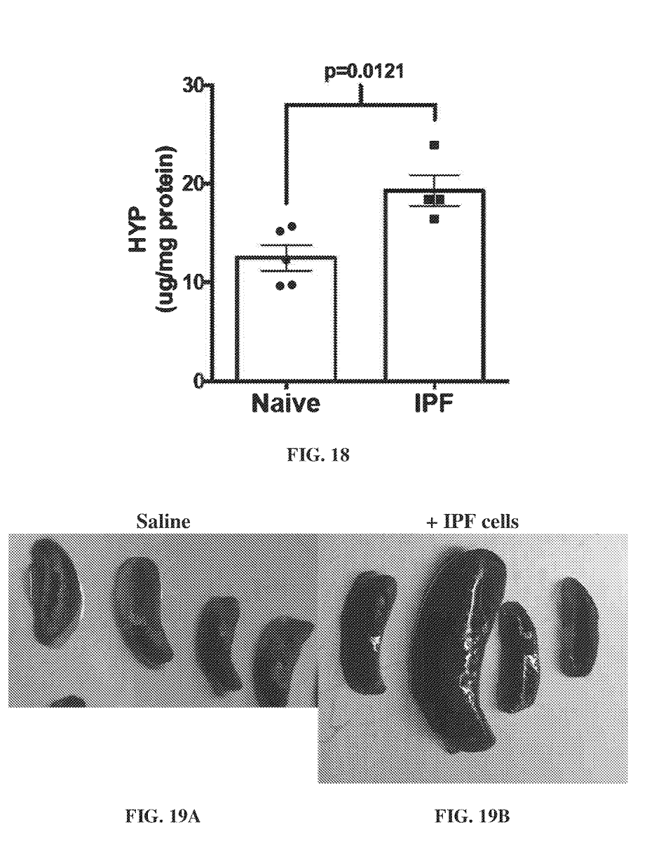

Histological analysis of fibrosis in SCID mice were performed 63 days after injection. FIG. 17A-FIG. 17B show lung tissue samples taken from naïve lungs (FIG. 17A) and IPF lungs (FIG. 17B). FIG. 17C-FIG. 17D show spleen tissue samples taken from naïve spleen (FIG. 17C) and IPF spleens (FIG. 17D). Hydroxyproline (fibrosis) levels in naïve versus IPF mice at day 63 are illustrated in FIG. 18. FIG. 19A-FIG. 19B show the spleen of mice 63 days after injection.

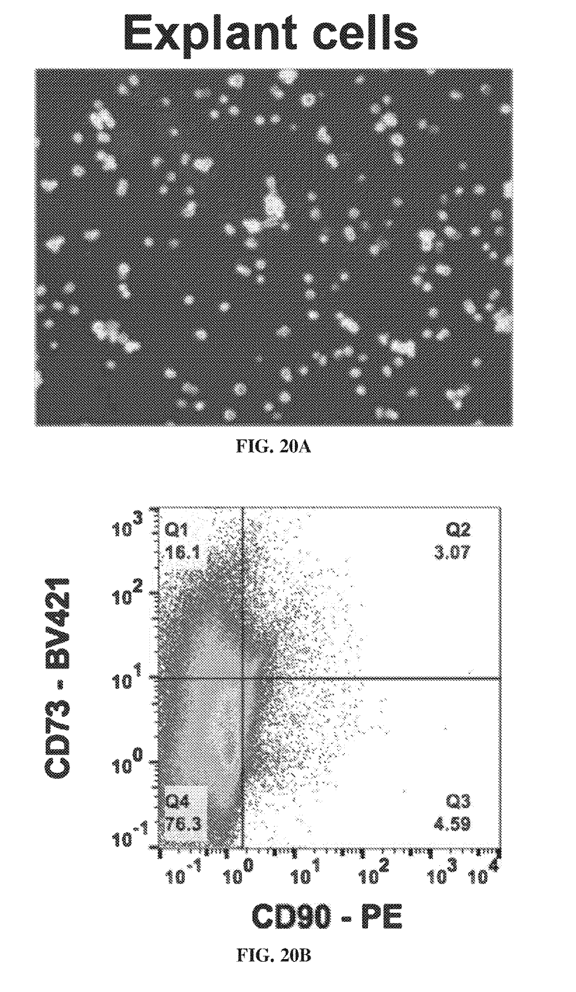

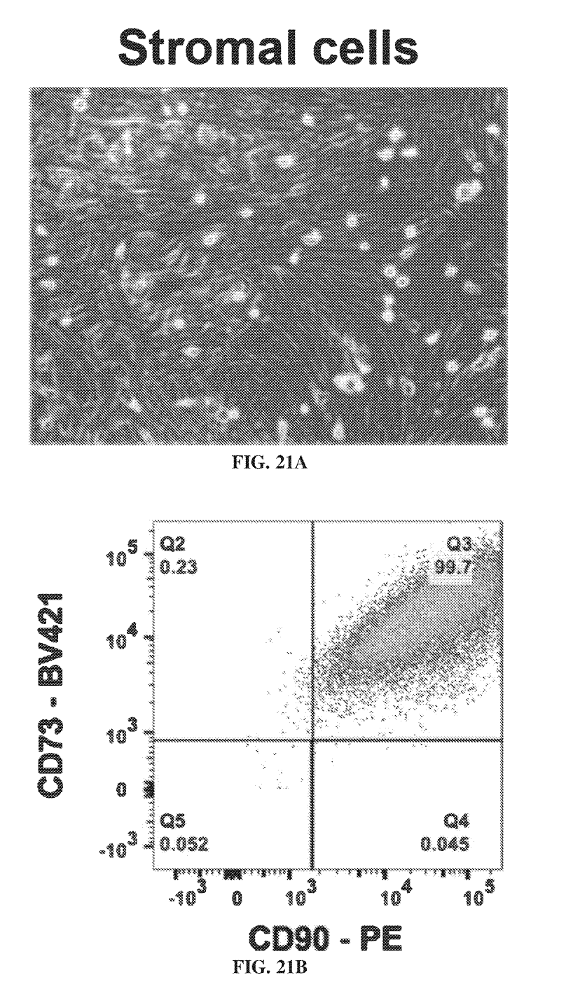

Histological analysis was performed on explant cells (FIG. 20A) and stromal cell (FIG. 21A). FIG. 20B shows the flow cytometry of CD90 versus CD73 markers for explant cells. FIG. 21B shows the flow cytometry of CD90 versus CD73 markers for stromal cells.

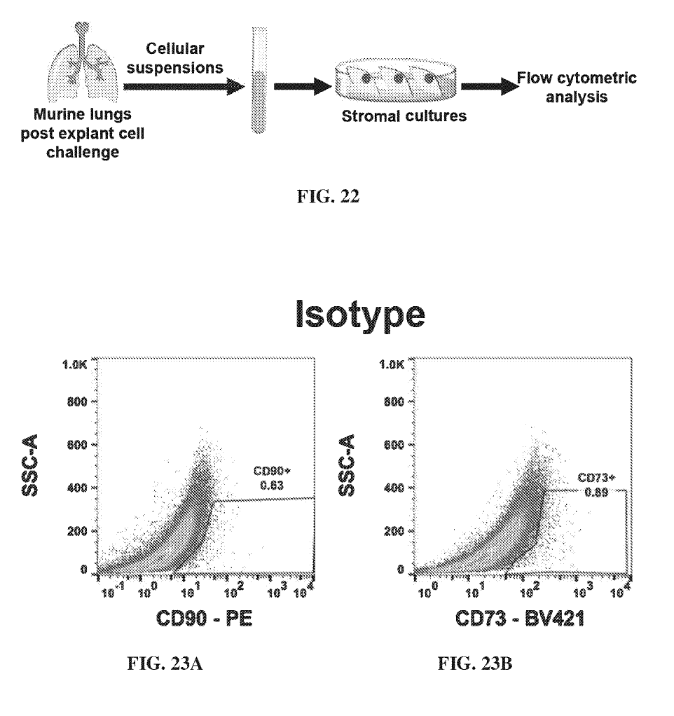

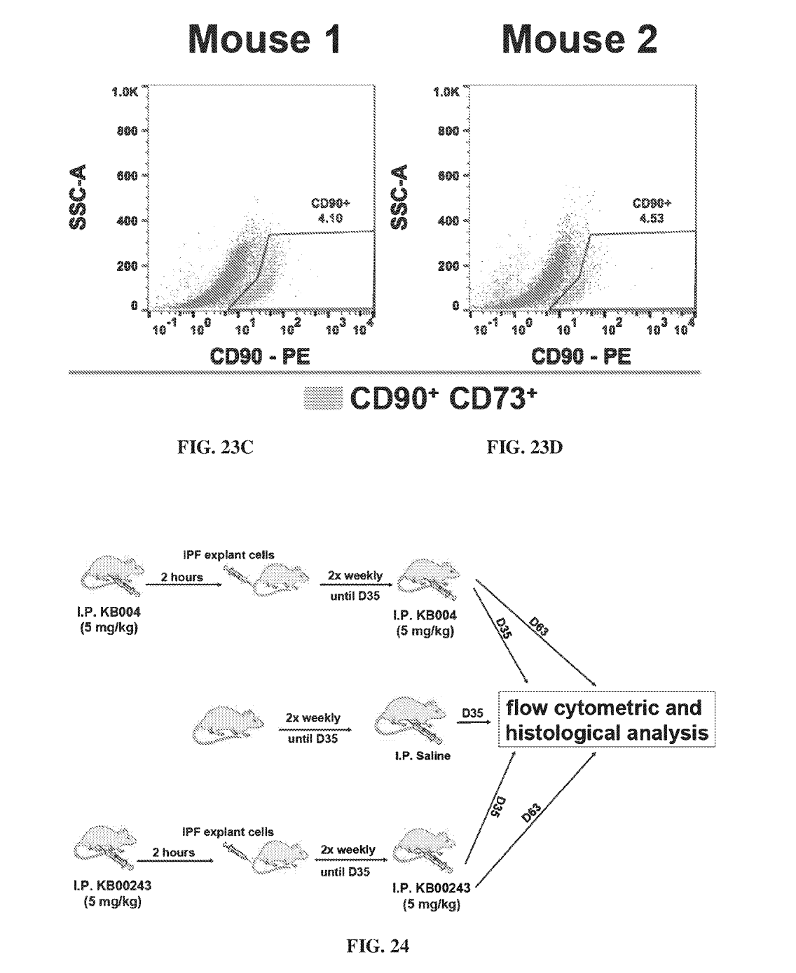

An experimental design is shown in FIG. 22. Murine lungs post explants cell challenge and held in cellular suspensions. The cellular suspensions were developed in stromal cultures and flow cytometric analysis was performed. FIG. 23A-FIG. 23B provide flow cytometry data showing the concentrations of CD90 (FIG. 23A) and CD73 (FIG. 23B) markers in the isotype cultures. FIG. 23C-FIG. 23D provide flow cytometry data showing the concentration of CD90 markers and CD73 markers in cultures from mouse 1 (FIG. 23C) and mouse 2 (FIG. 23D).

Efficacy of KB004 in a Humanized NOD/SCID Model of IPF

The experimental design for measuring the efficacy of KB004 as a preventative intervention in a humanized NOD/SCID model of IPF is shown in FIG. 24. Two groups of mice were separately intraperitoneally injected with KB004 and KB00243 at 5 mg/kg. Two hours after this injection, the mice were injected with IPF explanted cells and were subsequently injected with KB004 and KB00243 respectively twice a week until day 63. A control group of mice were injected twice weekly with saline until day 35. Flow cytometric and histological analysis was performed on days 35 and 63.

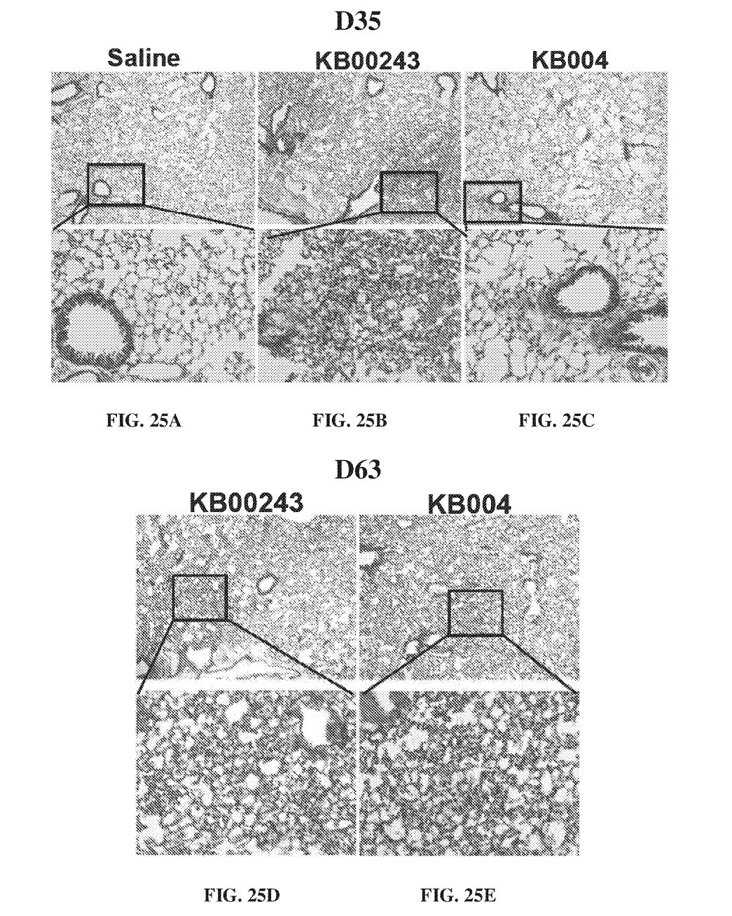

Histological analysis was performed on day 35 for mice that were administered saline (FIG. 25A), KB00243 (FIG. 25B), and KB004 (FIG. 25C). Histological analysis was performed on day 63 for mice that were administered KB00243 (FIG. 25D) and KB004 (FIG. 25E).

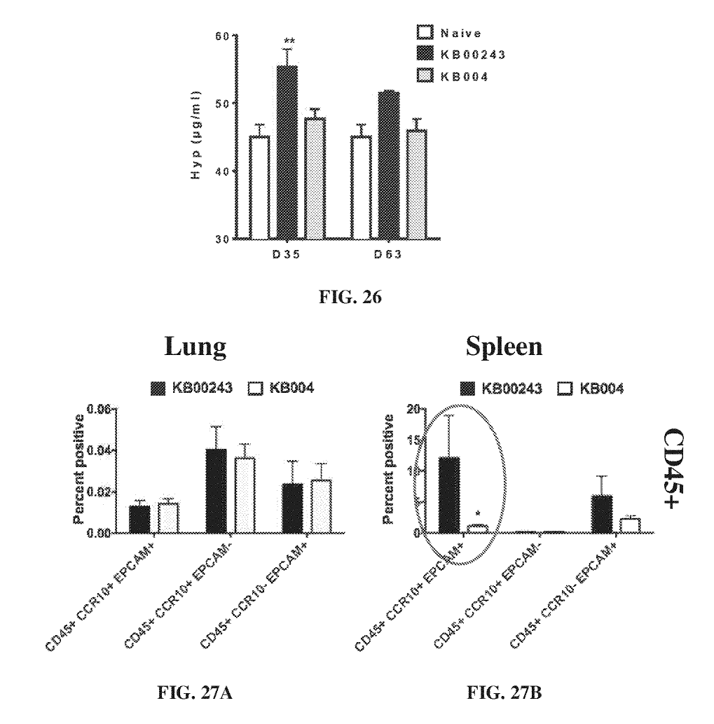

Hydroxyproline (fibrosis) levels on day 35 and day 63 in naïve mice and mice that were administered KB00243 and KB004 are illustrated in FIG. 26.

EphA3 cells were localized in the spleen at Day 35. KB004 targeted these cells. The percent of cells in the lung and spleen positive for CD45+ on day 35 are shown in FIG. 27A and FIG. 27B respectively. The percent positive for CD45+ CCR10+ EPCAM+ markers in the spleen for the mice administered with KB004 is greatly reduced when compared to the group administered with KB00243.

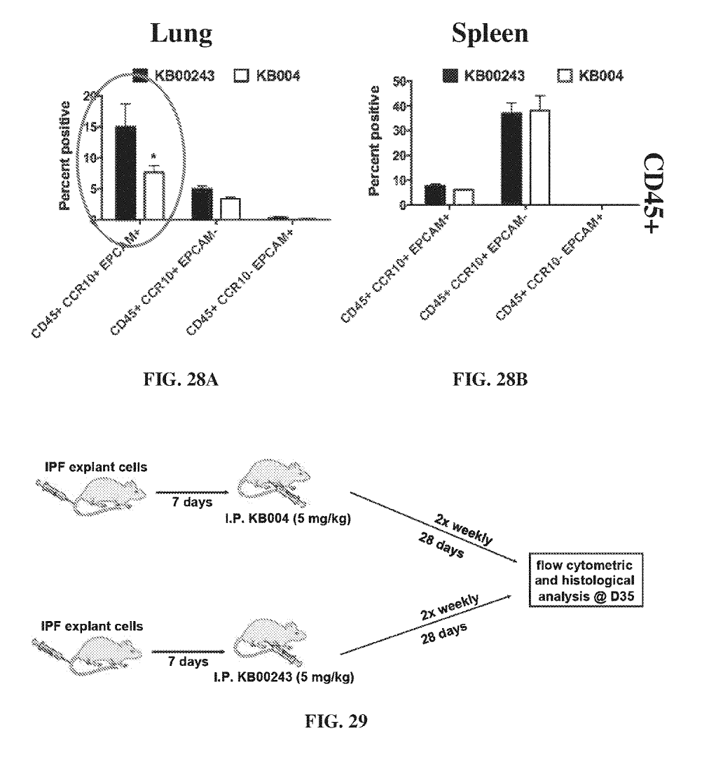

Epha3 cells were localized in the lung at Day 63. Prior KB004 treatment significantly reduced these cells in the lung. The percent of cells in the lung and spleen positive for CD45+ on day 35 are shown in FIG. 28A and FIG. 28B respectively. The percent positive for CD45+ CCR10+ EPCAM+ markers in the lung for the mice administered with KB004 is greatly reduced when compared to the group administered with KB00243.

The experimental design for measuring the efficacy of KB004 as a therapeutic intervention in a humanized NOD/SCID model of IPF is show in FIG. 29. Two groups of mice were injected with IPF explanted cells and then subsequently injected 7 days later with KB004 and KB00243 respectively twice a week for 28 days. Flow cytometric and histological analysis was performed on day 35.

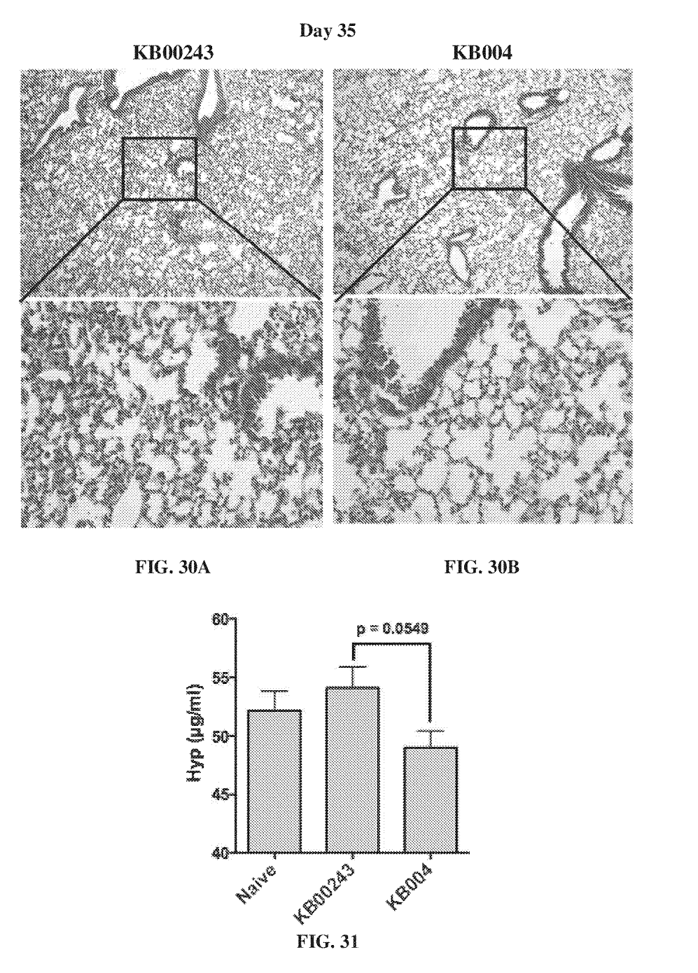

Histological analysis was performed on day 35 for mice that were administered saline KB00243 (FIG. 30A), and KB004 (FIG. 30B). Hydroxyproline (fibrosis) levels on day 35 in mice that were administered saline, KB00243, and KB004 are illustrated in FIG. 31.

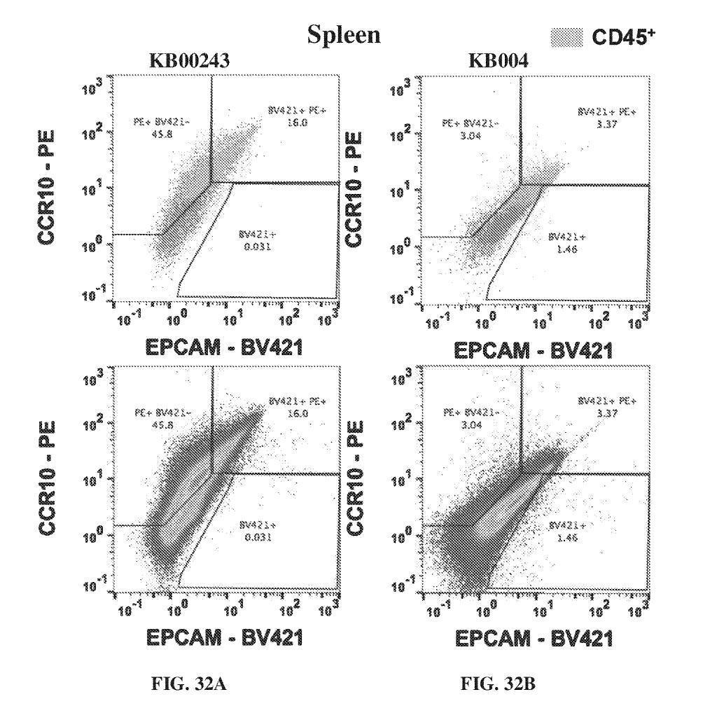

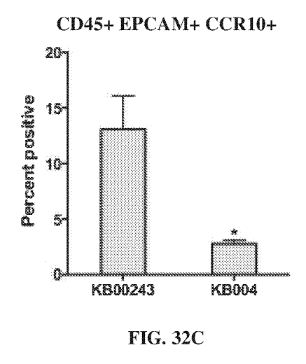

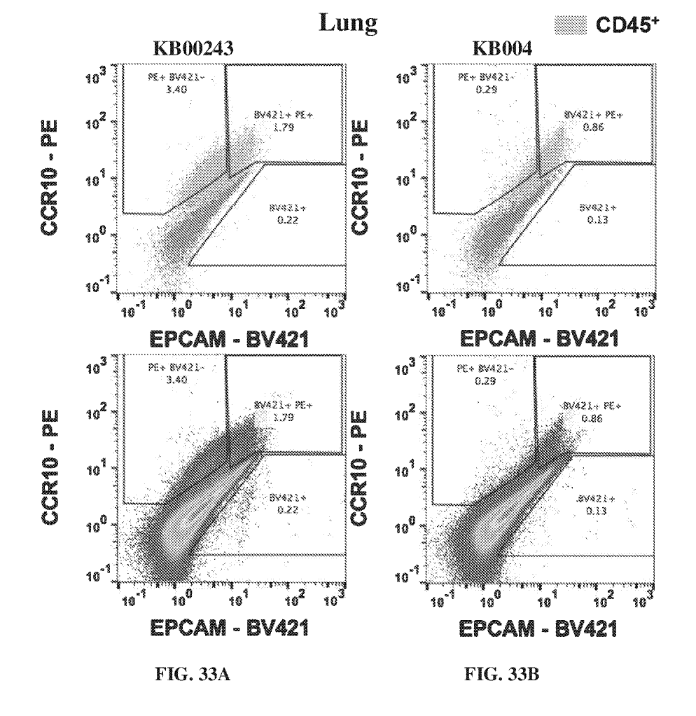

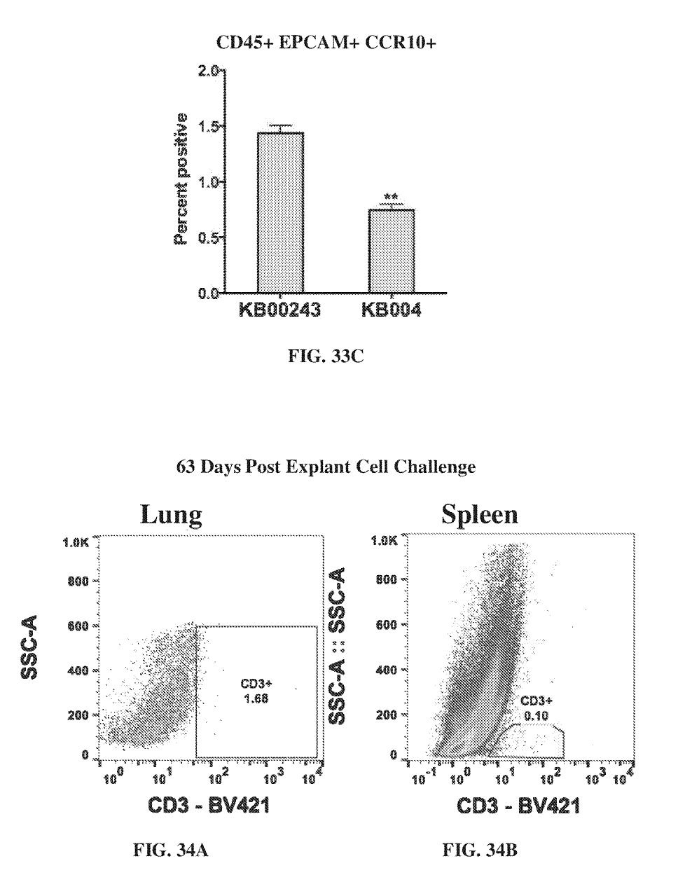

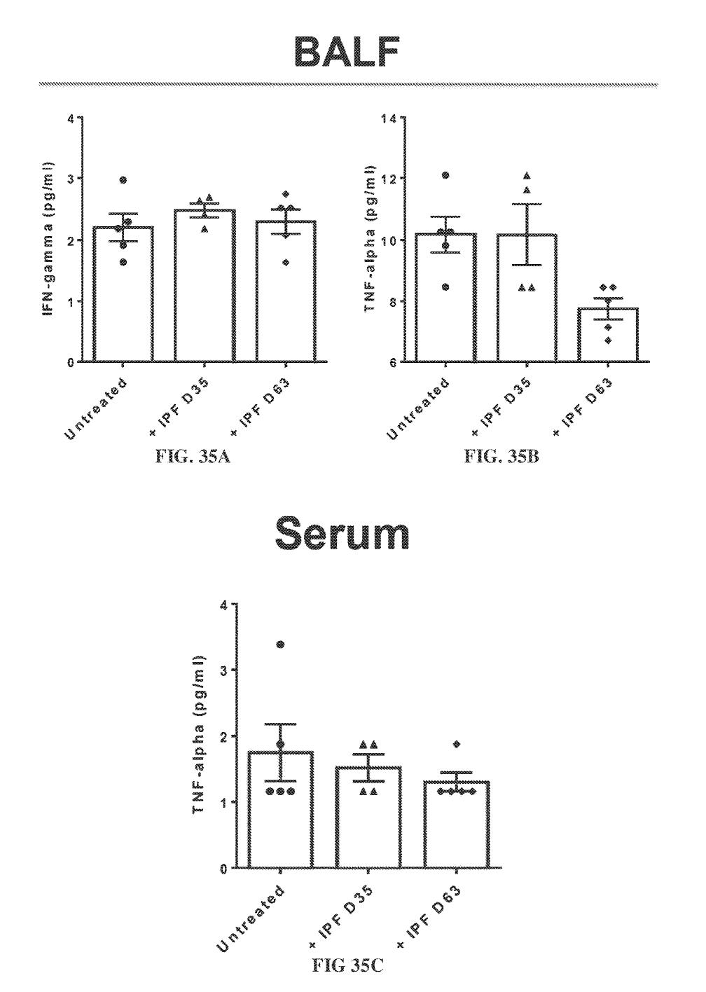

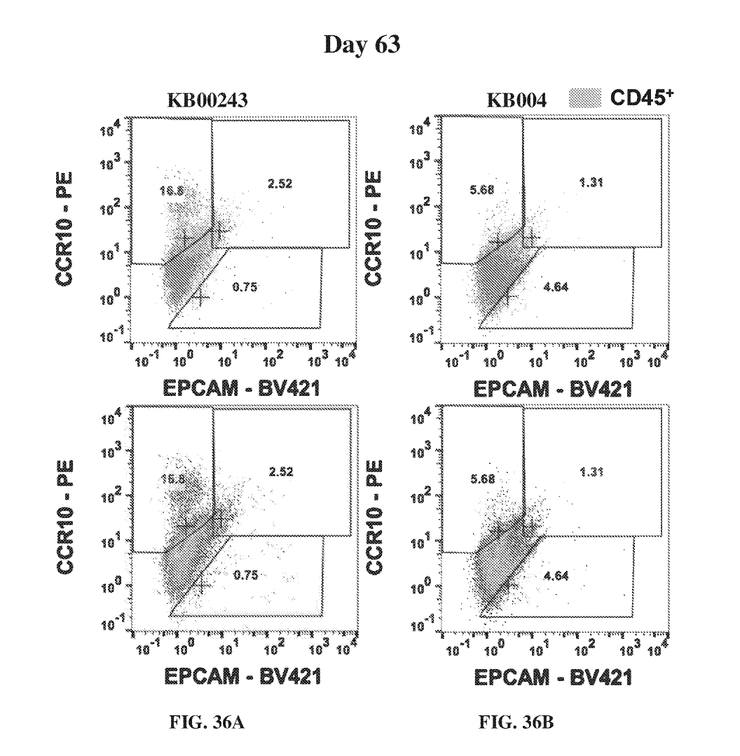

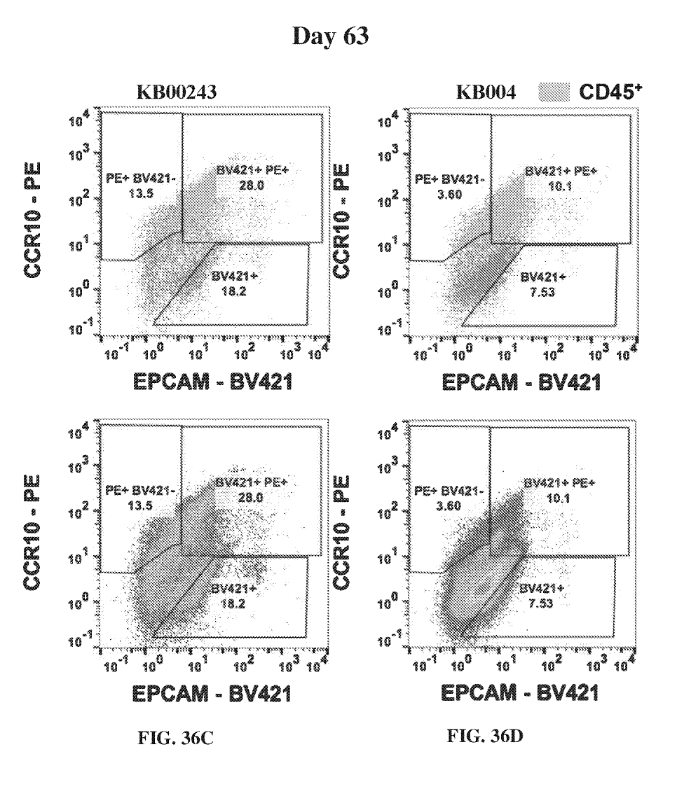

Flow cytometry analysis was performed showing the concentrations of EPCAM and CCR10 in the spleen of mice treated with KB00243 (FIG. 32A) and KB004 (FIG. 32B). Flow cytometry analysis was performed showing the concentrations of EPCAM and CCR10 in the lung of mice treated with KB00243 (FIG. 33A) and KB004 (FIG. 33B).

The percent of cells positive for CD45+ on day 35 are shown for the spleen (FIG. 32C) and lung (FIG. 33C).

IPF lungs contained EphA3 expressing stromal progenitor cells (SSEA4+, CD45+) that give rise to stromal cells in culture and induced pulmonary fibrosis in NOD/SCID mice. In the absence of immune cells, in vitro KB004 treatment enhanced IPF stromal cell differentiation. Targeting EphA3 expressing cells using an ADCC-inducing afucosylated antibody ameliorated the development of pulmonary fibrosis in NOD/SCID mice receiving xenografts of human IPF immune and nonimmune cells.

The pulmonary remodeling in NOD/SCIDS is not due to graft-versus-host disease (GVHD) (Covassin. L., et al. “Human peripheral blood CD4 T cell-engrafted non-obese diabetic-scid IL2rγnull H2-Ab1 tm1Gru Tg (human leucocyte antigen D-related 4) mice: a mouse model of human allogeneic graft-versus-host disease.” Clinical & Experimental Immunology 166.2 (2011): 269-280).

Few T cells were detected in the spleen but not the lungs of SCID mice 63 days post explant cell challenge. Flow cytometry was performed showing the concentrations of CD3 and SSC markers in the spleen (FIG. 34A) and lung (FIG. 34B).

KB004 Amino Acid Sequences

KB004 CDR sequences:
V_{H} region CDR1 (HCDR1):
(SEQ ID NO: 2)
GYWMN
V_{H} region CDR2 (HCDR2):
(SEQ ID NO: 3)
DIYPGSGNTNYDEKFQG
V_{H} region CDR3 (HCDR3):
(SEQ ID NO: 4)
GGYYEDFDS
V_{L} region CDR1 (LCDR1):
(SEQ ID NO: 5)
RASQGIISYL
V_{L} region CDR2 (LCDR2):
(SEQ ID NO: 6)
AASSLQS
V_{L} region CDR3 (LCDR3):
(SEQ ID NO: 7)
GQYANYPYT
KB004 Heavy chain signal sequence
(SEQ ID NO: 8)
MEWSWVFLFF LSVTTGVHS
KB004 Light chain signal sequence
(SEQ ID NO: 9)
MSVPTQVLGL LLLWLTDARC
KB004 V_{H} region sequence
(SEQ ID NO: 10)
QVQLVQSGAEVKKPGASVKVSCKASGYTFTGYWMNWVRQAPGQGLEWMGD
IYPGSGNTNYDEKFQGRVTMTRDTSISTAYMELSRLRSDDTAVYYCARGG
YYEDFDSWGQGTTVTVSS
KB004 V_{L} region sequence
(SEQ ID NO: 11)
DIQMTQSPSFLSASVGDRVTITCRASQGIISYLAWYQQKPEKAPKRLIYA
ASSLQSGVPSRFSGSGSGTEFTLTISSLQPEDFATYYCGQYANYPYTFGQ
GTKLEIK
KB004 Heavy chain sequence
(SEQ ID NO: 12)
QVQLVQSGAEVKKPGASVKVSCKASGYTFTGYWMNWVRQAPGQGLEWMGD
IYPGSGNTNYDEKFQGRVTMTRDTSISTAYMELSRLRSDDTAVYYCARGG
YYEDFDSWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
KB004 Kappa chain sequence
(SEQ ID NO: 13)
DIQMTQSPSFLSASVGDRVTITCRASQGIISYLAWYQQKPEKAPKRLIYA
ASSLQSGVPSRFSGSGSGTEFTLTISSLQPEDFATYYCGQYANYPYTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

While certain features disclosed herein have been illustrated and described herein, many modifications, substitutions, changes, and equivalents will now occur to those of ordinary skill in the art. It is, therefore, to be understood that the appended claims are intended to cover all such modifications and changes as fall within the true spirit disclosed herein.

1. A method of treating or preventing pulmonary fibrosis, the method comprising administering an effective amount of an anti-EphA3 antibody to a patient having pulmonary fibrosis or to a patient at risk for pulmonary fibrosis.

2. The method of claim 1, wherein the patient has idiopathic pulmonary fibrosis (IPF).

3. The method of claim 1, wherein the anti-EphA3 antibody induces ADCC, activates EphA3, and has the binding specificity of KB004.

4. The method of claim 1, wherein the anti-EphA3 antibody comprises:
(a) a V_{H} region that comprises:
(i) a CDR1 having a sequence GYWMN (SEQ ID NO: 2), a CDR2 having a sequence DIYPGS GNTNYDEKFQG (SEQ ID NO: 3), and a CDR3 having a sequence GGYYEDFDS (SEQ ID NO: 4); and
(b) a V_{L} region that comprises:
(i) a CDR1 having a sequence RASQGIISYL (SEQ ID NO: 5), a CDR2 having a sequence AASSLQS (SEQ ID NO: 6), and a CDR3 having a sequence GQYANYPYT (SEQ ID NO: 7).

5. The method of claim 1, wherein the anti-EphA3 antibody comprises a heavy chain signal sequence of SEQ ID NO: 8 and the light chain signal sequence of SEQ ID NO:9.

6. The method of claim 1, wherein the anti-EphA3 antibody comprises a V_{H} region comprising the sequence of SEQ ID NO: 10 and a V_{L} region comprising the sequence of SEQ ID NO: 11.

7. The method of claim 1, wherein the anti-EphA3 antibody comprises the heavy chain sequence of SEQ ID NO: 12 and the light chain sequence of SEQ ID NO: 13.

8. The method of claim 1, wherein the anti-EphA3 antibody comprises the heavy chain sequence of SEQ ID NO: 12 and the light chain sequence of SEQ ID NO: 13.

9. The method of claim 1, wherein the anti-EphA3 antibody comprises the heavy chain signal sequence of SEQ ID NO: 8 and the light chain signal sequence of SEQ ID NO: 9.

10. The method of claim 1, wherein the antibody is non-fucosylated.

11. The method of claim 1, wherein the anti-EphA3 antibody is administered preventatively.

12. The method of claim 1, wherein the anti-EphA3 antibody is administered therapeutically after the patient is diagnosed with pulmonary fibrosis.

13. The method of claim 1, wherein the anti-EphA3 antibody is administered to the patient twice weekly.

14. The method of claim 1, wherein the anti-EphA3 antibody is administered to the at a dose of 5 mg/kg.

15-28. (canceled)

29. A method of identifying a pulmonary fibrosis patient or a patient at risk for pulmonary fibrosis who is a candidate for treatment with KB004 or an antibody having the binding specificity of KB004, the method comprising evaluating bronchoalveolar lavage (BAL) fluid comprising fibroblasts obtained from the patient for EphA3 positivity on fibroblasts.

30. The method of claim 29, wherein the patient has idiopathic pulmonary fibrosis (IPF).

31. The method of claim 29, wherein the EphA3 positivity is determined by measuring the concentration of SSEA-4+, CD45+, CCR10+ markers.

32. The method of claim 29, wherein the evaluation step comprises immunohistochemistry or PCR.

33. A method of measuring the efficacy of treatment in a pulmonary fibrosis patient treated with KB004 or an antibody having the binding specificity of KB004, the method comprising evaluating the reduction of the disease markers of fibrosis.
